SEQUENCE LISTING (73 sequences)

SEQ ID NO: 1 - DNA, 25 bp, synthetic construct
  source 1..25: mol_type = other DNA
tgtgttaggtatggaagagattttt

SEQ ID NO: 2 - DNA, 80 bp, synthetic construct
  source 1..80: mol_type = other DNA
tgtgttaggtatggaagagattttttttttttttttttttttttttttttttttttttttaatcagccataccacatttg

SEQ ID NO: 3 - DNA, 80 bp, synthetic construct
  source 1..80: mol_type = other DNA
agagaatggataggttgtgtttttttttttttttttttttttttttttttttttttttttaatcagccataccacatttg

SEQ ID NO: 4 - DNA, 65 bp, synthetic construct
  source 1..65: mol_type = other DNA
tgtgttaggtatggaagagatttttttttttttttttttttttttaatcagccataccacatttg

SEQ ID NO: 5 - DNA, 20 bp, synthetic construct
  source 1..20: mol_type = other DNA
tttttttttttttttttttt

SEQ ID NO: 6 - DNA, 16 bp, synthetic construct
  source 1..16: mol_type = other DNA
gtaaaacgacggccag

SEQ ID NO: 7 - DNA, 20 bp, synthetic construct
  source 1..20: mol_type = other DNA
ggattattcataccgtccca

SEQ ID NO: 8 - DNA, 20 bp, synthetic construct
  source 1..20: mol_type = other DNA
caaatgtggtatggctgatt

SEQ ID NO: 9 - DNA, 15 bp, synthetic construct
  source 1..15: mol_type = other DNA
acctatccattctct

SEQ ID NO: 10 - DNA, 15 bp, synthetic construct
  source 1..15: mol_type = other DNA
acacatccattctct

SEQ ID NO: 11 - DNA, 15 bp, synthetic construct
  source 1..15: mol_type = other DNA
tccatacctatctct

SEQ ID NO: 12 - DNA, 15 bp, synthetic construct
  source 1..15: mol_type = other DNA
acacaacctatctct

SEQ ID NO: 13 - DNA, 15 bp, synthetic construct
  source 1..15: mol_type = other DNA
tccatacacatctct

SEQ ID NO: 14 - DNA, 15 bp, synthetic construct
  source 1..15: mol_type = other DNA
acctaacacatctct

SEQ ID NO: 15 - DNA, 15 bp, synthetic construct
  source 1..15: mol_type = other DNA
acctatctcttccat

SEQ ID NO: 16 - DNA, 15 bp, synthetic construct
  source 1..15: mol_type = other DNA
acacatctcttccat

SEQ ID NO: 17 - DNA, 15 bp, synthetic construct
  source 1..15: mol_type = other DNA
tctctacctatccat

SEQ ID NO: 18 - DNA, 15 bp, synthetic construct
  source 1..15: mol_type = other DNA
acacaacctatccat

SEQ ID NO: 19 - DNA, 15 bp, synthetic construct
  source 1..15: mol_type = other DNA
tctctacacatccat

SEQ ID NO: 20 - DNA, 15 bp, synthetic construct
  source 1..15: mol_type = other DNA
acctaacacatccat

SEQ ID NO: 21 - DNA, 15 bp, synthetic construct
  source 1..15: mol_type = other DNA
tccattctctaccta

SEQ ID NO: 22 - DNA, 15 bp, synthetic construct
  source 1..15: mol_type = other DNA
acacatctctaccta

SEQ ID NO: 23 - DNA, 15 bp, synthetic construct
  source 1..15: mol_type = other DNA
tctcttccataccta

SEQ ID NO: 24 - DNA, 15 bp, synthetic construct
  source 1..15: mol_type = other DNA
acacatccataccta

SEQ ID NO: 25 - DNA, 15 bp, synthetic construct
  source 1..15: mol_type = other DNA
tctctacacaaccta

SEQ ID NO: 26 - DNA, 15 bp, synthetic construct
  source 1..15: mol_type = other DNA
tccatacacaaccta

SEQ ID NO: 27 - DNA, 15 bp, synthetic construct
  source 1..15: mol_type = other DNA
tccattctctacaca

SEQ ID NO: 28 - DNA, 15 bp, synthetic construct
  source 1..15: mol_type = other DNA
acctatctctacaca

SEQ ID NO: 29 - DNA, 15 bp, synthetic construct
  source 1..15: mol_type = other DNA
tctcttccatacaca

SEQ ID NO: 30 - DNA, 15 bp, synthetic construct
  source 1..15: mol_type = other DNA
acctatccatacaca

SEQ ID NO: 31 - DNA, 15 bp, synthetic construct
  source 1..15: mol_type = other DNA
tctctacctaacaca

SEQ ID NO: 32 - DNA, 15 bp, synthetic construct
  source 1..15: mol_type = other DNA
tccatacctaacaca

SEQ ID NO: 33 - DNA, 20 bp, synthetic construct
  source 1..20: mol_type = other DNA
tccattctcttgggacggta

SEQ ID NO: 34 - DNA, 20 bp, synthetic construct
  source 1..20: mol_type = other DNA
acctatctcttgggacggta

SEQ ID NO: 35 - DNA, 20 bp, synthetic construct
  source 1..20: mol_type = other DNA
acacatctcttgggacggta

SEQ ID NO: 36 - DNA, 20 bp, synthetic construct
  source 1..20: mol_type = other DNA
tctcttccattgggacggta

SEQ ID NO: 37 - DNA, 20 bp, synthetic construct
  source 1..20: mol_type = other DNA
acctatccattgggacggta

SEQ ID NO: 38 - DNA, 20 bp, synthetic construct
  source 1..20: mol_type = other DNA
acacatccattgggacggta

SEQ ID NO: 39 - DNA, 20 bp, synthetic construct
  source 1..20: mol_type = other DNA
tctctacctatgggacggta

SEQ ID NO: 40 - DNA, 20 bp, synthetic construct
  source 1..20: mol_type = other DNA
tccatacctatgggacggta

SEQ ID NO: 41 - DNA, 20 bp, synthetic construct
  source 1..20: mol_type = other DNA
acacaacctatgggacggta

SEQ ID NO: 42 - DNA, 20 bp, synthetic construct
  source 1..20: mol_type = other DNA
tctctacacatgggacggta

SEQ ID NO: 43 - DNA, 20 bp, synthetic construct
  source 1..20: mol_type = other DNA
tccatacacatgggacggta

SEQ ID NO: 44 - DNA, 20 bp, synthetic construct
  source 1..20: mol_type = other DNA
acctaacacatgggacggta

SEQ ID NO: 45 - DNA, 10 bp, synthetic construct
  source 1..10: mol_type = other DNA
agagaagaga

SEQ ID NO: 46 - DNA, 10 bp, synthetic construct
  source 1..10: mol_type = other DNA
aaggtttgga

SEQ ID NO: 47 - DNA, 15 bp, synthetic construct
  source 1..15: mol_type = other DNA
agagatgtgtagaga

SEQ ID NO: 48 - DNA, 15 bp, synthetic construct
  source 1..15: mol_type = other DNA
tgtgttgtgtttgga

SEQ ID NO: 49 - DNA, 10 bp, synthetic construct
  source 1..10: mol_type = other DNA
agagaatgga

SEQ ID NO: 50 - DNA, 10 bp, synthetic construct
  source 1..10: mol_type = other DNA
taggtttgga

SEQ ID NO: 51 - DNA, 15 bp, synthetic construct
  source 1..15: mol_type = other DNA
agagatgtgtatgga

SEQ ID NO: 52 - DNA, 15 bp, synthetic construct
  source 1..15: mol_type = other DNA
tgtgtatggattgga

SEQ ID NO: 53 - DNA, 20 bp, synthetic construct
  source 1..20: mol_type = other DNA
agagatgtgtatggaagaga

SEQ ID NO: 54 - DNA, 25 bp, synthetic construct
  source 1..25: mol_type = other DNA
agagatgtgttgtgttttttttttt

SEQ ID NO: 55 - DNA, 25 bp, synthetic construct
  source 1..25: mol_type = other DNA
ttttttttttttttttttttttttt

SEQ ID NO: 56 - DNA, 15 bp, synthetic construct
  source 1..15: mol_type = other DNA
acacatctcttctct

SEQ ID NO: 57 - DNA, 25 bp, synthetic construct
  source 1..25: mol_type = other DNA
acacaacacatctcttctcttctct

SEQ ID NO: 58 - DNA, 30 bp, synthetic construct
  source 1..30: mol_type = other DNA
tgtgttaggtatggaagagatttttttttt

SEQ ID NO: 59 - DNA, 65 bp, synthetic construct
  source 1..65: mol_type = other DNA
tgtgttaggtatggaagagattttttttttttttttttttttttttttttttttttttttttttt

SEQ ID NO: 60 - DNA, 65 bp, synthetic construct
  source 1..65: mol_type = other DNA
agagaatggataggttgtgtaatcagccataccacatttgttttttttttttttttttttttttt

SEQ ID NO: 61 - DNA, 20 bp, synthetic construct
  source 1..20: mol_type = other DNA
agagaatggataggttgtgt

SEQ ID NO: 62 - DNA, 20 bp, synthetic construct
  source 1..20: mol_type = other DNA
atggaagagatgtgttaggt

SEQ ID NO: 63 - DNA, 20 bp, synthetic construct
  source 1..20: mol_type = other DNA
taggttgtgtagagaatgga

SEQ ID NO: 64 - DNA, 20 bp, synthetic construct
  source 1..20: mol_type = other DNA
tgtgttaggtatggaagaga

SEQ ID NO: 65 - DNA, 15 bp, synthetic construct
  source 1..15: mol_type = other DNA
tccattctcttccat

SEQ ID NO: 66 - DNA, 20 bp, synthetic construct
  source 1..20: mol_type = other DNA
aatcagccataccacatttg

SEQ ID NO: 67 - DNA, 20 bp, synthetic construct
  source 1..20: mol_type = other DNA
gatccgattggaaccgtccc

SEQ ID NO: 68 - DNA, 71 bp, synthetic construct
  source 1..71: mol_type = other DNA
acgagcatcagcagcatacgannnnnnnnnnnnnnnnnnnntttttttttttttttttttttttttttttt

SEQ ID NO: 69 - DNA, 72 bp, synthetic construct
  source 1..72: mol_type = other DNA
agagaatggataggttgtgtnnnnnnnnnnnnnnnnnnnnttttttttttttttttttttttttttttttvn

SEQ ID NO: 70 - DNA, 30 bp, synthetic construct
  source 1..30: mol_type = other DNA
  misc_feature 28..29: note = RNA
  misc_feature 1..27: note = DNA
  misc_feature 30: note = DNA
aagcagtggtatcaacgcagagtacatggg

SEQ ID NO: 71 - DNA, 34 bp, synthetic construct
  source 1..34: mol_type = other DNA
gtctcgtgggctcggagatgtgtataagagacag

SEQ ID NO: 72 - DNA, 54 bp, synthetic construct
  source 1..54: mol_type = other DNA
tcgtcggcagcgtcagatgtgtataagagacagacgagcatcagcagcatacga

SEQ ID NO: 73 - DNA, 21 bp, synthetic construct
  source 1..21: mol_type = other DNA
acgagcatcagcagcatacga

(19) United States
(12) Patent Application Publication
(10) Pub. No.: US 2026/0327683 A1
(43) Pub. Date: Oct. 22, 2026

(54) MULTIPLEXED OPTICAL BARCODING FOR SPATIAL OMICS

(71) Applicant: President and Fellows of Harvard College, Cambridge, MA (US)

(72) Inventors: Xiaowei Zhuang, Cambridge, MA (US); Aditya Venkatramani, Cambridge, MA (US)

(73) Assignee: President and Fellows of Harvard College, Cambridge, MA (US)

(21) Appl. No.: 19/474,064

(22) PCT Filed: Apr. 10, 2024
(86) PCT No.: PCT/US2024/023842

§ 371 Date: Oct. 9, 2025

Related U.S. Application Data
(60) Provisional application No. 63/458,610, filed on Apr. 11, 2023.
(60) Provisional application No. 63/506,294, filed on Jun. 5, 2023.
(60) Provisional application No. 63/506,337, filed on Jun. 5, 2023.

(51) Int. Cl. C12Q 1/6806; C12Q 1/6869

(52) U.S. Cl. CPC C12Q 1/6806; C12Q 1/6869

(57) ABSTRACT

The present disclosure generally relates to systems and methods for spatially barcoding or identifying molecules in a sample. Certain embodiments are directed to multidimensional spatial omics tools with targeted or untargeted biomolecule, highly parallelized barcoding, and sub-cellular resolution. Some aspects may introduce spatial barcodes to biomolecules using light and sequence them to decipher their content and position inside cells and blocks of tissue. The spatial barcoding can be achieved using light beams that can address pixels in a 3D space without having any dead space between them. Having these features of untargeted RNA measurement, 3D profiling of tissues, parallelized barcoding, no dead space, and the possibility of single-cell sequencing provides new ways to collect molecular and spatial information from tissues, e.g., for discovery in cancer biology, immunology, neuroscience, and developmental biology. In certain cases, having an untargeted, fast, and/or 3D spatial omics technology like the ones described herein can facilitate discovery, e.g., for studying spatial organization of molecular features of tissues, organs, and organisms, etc., which may allow their study in healthy, diseased, drugged state, or the like.

### RELATED APPLICATIONS

This application claims the benefit of U.S. Provisional Patent Application Ser. No. 63/458,610, filed Apr. 11, 2023, entitled “Multiplexed Optical Barcoding for Spatial Omics,” by Zhuang, et al.; U.S. Provisional Patent Application Ser. No. 63/506,294, filed Jun. 5, 2023, entitled “Multiplexed Optical Barcoding for Spatial Omics,” by Zhuang, et al.; and U.S. Provisional Patent Application Ser. No. 63/506,337, filed Jun. 5, 2023, entitled “Chemical Ligation Techniques,” by Zhuang, et al., each of which is incorporated herein by reference in its entirety.

### GOVERNMENT FUNDING

This invention was made with government support under NS116593 awarded by National Institutes of Health (NIH). The government has certain rights in this invention.

### FIELD

The present disclosure generally relates to systems and methods for spatially barcoding or identifying molecules in a sample.

### BACKGROUND

Spatial omics is a new frontier in understanding how gene activity controls and manifests in the working of tissues and organs. It was named the method of the year 2020 by Nature Methods and has been approached using methods built on in-situ hybridization, in-situ sequencing or spatially-resolved capture followed by next-generation sequencing. These methods have been used to study a diverse set of systems in neuroscience, developmental biology, cancer biology, etc. Such methods have been used to create reference cell atlases of the brain, mouse embryo, cancer tissues and tumor microenvironment, plant leaf, and many others. These spatial maps with cellular resolution can further be used to decipher cell-cell interactions and underlying molecular mechanisms. Brain, tumor microenvironments, and embryos are inherently three-dimensional with high levels of cellular heterogeneity and methods to study them in three-dimensions can further the understanding of molecular mechanisms in such systems. With spatial omics still developing and applications being identified, new tools for studying spatial organization of molecular features of tissues, organs, and organisms are needed.

### SUMMARY

The present disclosure generally relates to systems and methods for spatially barcoding or identifying molecules in a sample. The subject matter of the present disclosure involves, in some cases, interrelated products, alternative solutions to a particular problem, and/or a plurality of different uses of one or more systems and/or articles.

One aspect is generally drawn to a composition. In one set of embodiments, the composition comprises a plurality of nucleic acids, encoding information about their spatial positions within a sample.

In accordance with another set of embodiments, the composition comprises a first nucleic acid attached to a sample in a first location, and a second nucleic acid attached to the sample in a second location neighboring the first location. In some cases, the first nucleic acid and the second nucleic acid each comprise a stopper sequence between encoding sequences.

In one set of embodiments, the composition comprises a plurality of DNA sequences encoding 2- or 3-dimension information about their location within a sample.

In another set of embodiments, the composition comprises a plurality of DNA sequences encoding 3-dimension information about their location within a sample.

In addition, certain aspects are generally drawn to methods. For example, in one set of embodiments, the method comprises applying a first beam of light to a first location in a sample to attach a first nucleic acid to the sample within the first location, and applying a second beam of light to a second location in the sample to attach a second nucleic acid to the sample within the second location. In some cases, the first location and the second location overlap at at least one location within the sample.

The method, in accordance with another set of embodiments, comprises applying a first beam of light to a first location in a sample to attach a first nucleic acid to the sample within the first location, and applying a second beam of light to a second location in the sample to attach a second nucleic acid to the sample within the second location such that at at least one spatial position within the sample, the second nucleic acid is attached to the first nucleic acid.

In still another set of embodiments, the method comprises applying beams of light to a sample to attach nucleic acids to the sample, each beam of light applied to attach a nucleic acid to attached nucleic acids in the sample to form barcodes comprising a plurality of the nucleic acids.

The method, in yet another set of embodiments, comprises providing a sample comprising nucleic acids comprising a first sequence, a photocleavable linker, and a spacer sequence; applying light to a location in the sample to cleave the photocleavable linker and remove the spacer sequence from the nucleic acids in the location; and attaching a second sequence to the nucleic acid using a DNA ligase, wherein the spacer sequence, when present, inhibits the DNA-ligase from attaching nucleic acids.

In another set of embodiments, the method comprises applying beams of light to a sample to attach nucleic acids to different spatial positions within a sample. In some cases, the nucleic acids at different spatial positions within the sample comprise distinguishable sequences.

The method, in accordance with yet another set of embodiments, comprises applying beams of light to a sample to attach nucleic acids to different spatial positions within a sample, in certain embodiments, the nucleic acids encode their spatial positions within the sample.

According to still another set of embodiments, the method comprises sequencing nucleic acids taken from a sample, and determining spatial positions of the nucleic acids within the sample based on spatial information encoded therein.

The method, in another set of embodiments, comprises sequencing nucleic acids taken from a sample, and constructing an image based on spatial information encoded within the nucleic acids.

In one set of embodiments, the method comprises defining spatial positions within a sample encoded by nucleic acids, and forming barcodes at the spatial positions within the sample that encodes the spatial positions.

In another set of embodiments, the method comprises defining a plurality of spatial positions in a sample as intersections of beams of light applied from at least 2 different angles, defining distinguishable nucleic acid sequences identifying at least some of the spatial positions, and forming nucleic acids within at least some of the spatial positions that correspond to the defined nucleic acid sequences that identifies the respective spatial positions.

The method, in yet another set of embodiments, comprises sequencing nucleic acids encoding information about their spatial positions in a sample, where the information comprises encoding sequences interspersed with stopper sequences; and rejecting encoding sequences based on their positions relative to the stopper sequences.

In one set of embodiments, the method comprises exposing a sample to DNA tags; applying light to specific locations within the sample to cause DNA letters to append onto the DNA tags within the sample at the specific locations; extracting the DNA tags with appended letters; repeating the exposing, applying, and extracting steps at least one or more times; and sequencing the appended DNA tags from the sample.

The method, in another set of embodiments, comprises applying light to append a DNA letter onto a DNA tag, and repeating the appending of DNA letters to create a stack of letter (or barcode) on the DNA tag.

According to yet another set of embodiments, the method comprises applying light to a first set of locations within a sample to cause a DNA letter to append to DNA tags within the sample at the specific locations; applying light to a second set of locations within the sample to cause a second DNA tag to append to DNA tags within the sample at the specific locations, wherein the second DNA letter is different from the first DNA letter, and wherein at least some of the second set of locations are different locations within the sample than the first set of locations; and sequencing the DNA tags with the first and/or the second letter from the sample.

The method, in still another set of embodiments, comprises applying light to a first set of locations within a sample to cause a DNA letter to append onto DNA tags within the sample at the specific locations; applying light to a second set of locations within the sample to cause a second DNA letter to append onto DNA tags within the sample at the specific locations, wherein the second DNA letter can be the same or different from the first DNA letter, and wherein at least some of the second set of locations are different locations within the sample than the first set of locations; repeating the application of light and appending over various sets of locations; extracting the DNA tags with appended DNA letters; and sequencing the extracted DNA tags with appended DNA letters.

In yet another set of embodiments, the method comprises applying light to a first set of locations within a sample to cause a DNA letter to append onto DNA tags within the sample at the specific locations; applying light to the next set of locations within a sample to cause another DNA letter to append onto DNA tags within the sample at the specific locations; wherein the locations are different from the previous set of locations and the DNA letter is different from the previous ones; repeating the application of light until all desired locations have one DNA letter appended to DNA tag; repeating the previous three steps (called a round) to append additional DNA letters onto DNA tags to form a barcode, wherein DNA letters between each round of appending can be the same or different and light can be applied on the same or different set of locations; extracting the DNA tags with appended barcodes; and sequencing the extracted DNA tags with appended barcodes; In one set of embodiments, the method comprises ligating a oligonucleotide with a photocleaveable linker onto another oligonucleotide using a splint; using a plurality of splints to parallelize ligation; photocleaving the linker to allow another round of ligation; and repeating the previous three steps for one or more rounds of ligation.

The method, in another set of embodiments, comprises synthesizing DNA using nucleotides bound to TdT enzyme with a photocleaveable linker to define spatial barcodes.

In yet another set of embodiments, the method comprises synthesizing DNA using nucleosides with photolabile groups to define spatial barcodes.

In still another set of embodiments, the method comprises ligating DNA using oligonucleotides with a photocleaveable linker to define spatial barcodes.

According to another set of embodiments, the method comprises designing splints for ligation to be robust to errors in ligation based DNA letter appending; and designing DNA letters with a spacer sequence with a photocleaveable linker to be robust to errors in DNA letter appending.

In yet another set of embodiments, the method comprises applying beams of light to a sample at 3 different angles, to define voxels at their intersection within the sample; defining a barcode for each voxel, wherein the barcode may consist of one or more DNA letters and the barcode may be unique or the same between voxels; applying light to a first set of locations or angles within a sample to cause a first DNA letter to append onto DNA tags within the sample where it is or has been illuminated; applying light to the next set of locations or angles within a sample to cause another DNA letter to append onto DNA tags within the sample where it is or has been illuminated; where the DNA letter may be the same or different from the previous one and the locations or angles may be the same or different from the previous one; and repeating the application of light until all desired voxels have the correspondingly defined barcode.

The method, in still another set of embodiments, comprises appending additional DNA letters at specific positions within the barcode to define an error detecting code; and determining errors from sequenced barcodes using the defined code.

According to yet another set of embodiments, the method comprises appending additional DNA letters at specific positions within the barcode to define an error detecting code; determining errors from sequenced barcodes using the defined code; and performing error correction on detected errors.

In one set of embodiments, the method comprises designing DNA letters and barcodes to detect errors; and determining errors from sequenced barcodes using the defined code.

In another set of embodiments, the method comprises designing DNA letters and barcodes to detect errors; determining errors from sequenced barcodes using the defined code; and performing error correction on detected errors.

According to still another set of embodiments, the method comprises sequencing a plurality of DNA; extracting 2- or 3-dimensional positional information about the DNA based on its sequence; and constructing an image based on the 2- or 3-dimensional positional information indicating the location of molecules within a sample.

In one set of embodiments, the method comprises exposing a sample to DNA tags; applying light to specific locations within the sample to cause the DNA tags to bind to molecules within the sample at the specific locations; removing the DNA tags; repeating the exposing, applying, and removing steps at least one or more times; and sequencing the bound DNA tags from the sample.

In another set of embodiments, the method comprises applying light to a first set of locations within a sample to cause a first DNA tag to bind to molecules within the sample at the specific locations; applying light to a second set of locations within the sample to cause a second DNA tag to bind to molecules within the sample at the specific locations, wherein the second DNA tag is different from the first DNA tag, and wherein at least some of the second set of locations are different locations within the sample than the first set of locations; and sequencing the first and second DNA tags from the sample.

The method, in yet another set of embodiments, comprises applying 3 beams of light to a sample from a single direction, wherein the beams of light are applied at different angles, to define voxels within the sample; and causing binding of DNA within the sample at locations where the 3 beams of light intersect.

In still another set of embodiments, the method comprises sequencing a plurality of DNA; extracting 3-dimensional positional information about the DNA based on its sequence; and constructing an image based on the 3-dimensional positional information indicating the location of molecules within a sample.

In one embodiment, the method comprises applying beams of light to a sample to cause binding of DNA to the sample at locations where the beams intersect.

In still another embodiment, the method comprises applying a beam of light to a portion of a sample to attach a nucleic acid sequence to the sample within the portion; and repeating the applying step one or more times to attach nucleic acid sequences to the sample, wherein at least two of the attached nucleic acid sequences are distinguishable.

In yet another embodiment, the method comprises applying a beam of light to a portion of a sample to attach a nucleic acid sequence to the sample within the portion; and repeating the applying step one or more times to attach nucleic acid sequences to the sample, wherein at least two of the attached nucleic acid sequences are attached to each other.

In one embodiment, the method comprises applying beams of light to a sample to cause binding of a first nucleic acid at a location where the beams of light intersect, and attaching a second nucleic acid to the first nucleic acid to produce a barcode. In another embodiment, the method comprises attaching nucleic acids to different spatial positions within a sample by applying beams of light to the sample to cause the binding of the nucleic acids at spatial positions within the sample wherein the beams of light intersect.

In another aspect, the present disclosure encompasses methods of making one or more of the embodiments described herein, for example, systems and methods for spatially barcoding or identifying molecules in a sample. In still another aspect, the present disclosure encompasses methods of using one or more of the embodiments described herein, for example, systems and methods for spatially barcoding or identifying molecules in a sample.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 is a schematic illustrating tagging of mRNA and/or proteins, spatial barcoding, extraction, and sequencing, in one embodiment;

FIG. 2 is a schematic illustrating introducing DNA tags into a sample, in another embodiment;

FIG. 3 illustrates an example 2-dimensional region of space has been discretized, in yet another embodiment;

FIG. 4 is a schematic illustrates barcodes uniquely mapping different spatial positions within a sample, in still another embodiment;

FIG. 5 illustrates a recursive algorithm for generating barcodes, in yet another embodiment;

FIG. 6 is an assay showing ligation and photocleaving efficiency, in one embodiment;

FIG. 7 is an assay showing ligation of a DNA tag, in another embodiment;

FIG. 8 is a histogram showing melting temperatures of splints, in still another embodiment;

FIG. 9 is shows a ligation assay, in yet another embodiment;

FIG. 10 shows an assay used for determining efficiency, in still another embodiment;

FIGS. 11A-11C shows light applied to a sample to define various voxels, in one embodiment;

FIGS. 12A-12C illustrate various error corrections, in another embodiment;

FIG. 13 illustrates the use of various stopper sequences, in yet another embodiment;

FIGS. 14A-14B illustrate a simulation using alternate sites and stopper sequences, in still another embodiment;

FIGS. 15A-15F illustrates the attachment of nucleic acids to different spatial positions within a sample, in yet another embodiment;

FIG. 16 illustrates the tapestation results for 1, 2, 3, and 4 ligations, with >95% efficiency per ligation, in one embodiment;

FIG. 17 is an image showing the fluorescent signal in the photocleaved region, in another embodiment;

FIG. 18 is a DAPI signal showing the presence of cells, in yet another embodiment;

FIGS. 19A-19B show the mRNA content of tissue samples captured using in-situ Reverse Transcription (RT), in still another embodiment;

FIG. 20 is a schematic illustrating a ligation order, in one embodiment;

FIG. 21 shows a DAPI image, in another embodiment; and

FIGS. 22A-22B illustrate results of sequencing mapping onto mouse and human genomes, in yet another embodiment.

### BRIEF DESCRIPTION OF THE SEQUENCES

Below is a list of DNA sequences used for the embodiments and examples herein.

| | | SEQ ID |
|---|---|---|
| Type | Sequence | NO: |
| DNA tag | /5Phos/TGTGT TAGGT ATGGA AGAGA TTTTT/36-FAM/ | 1 |
| DNA tag | /5Phos/TGTGT TAGGT ATGGA AGAGA TTTTT TTTTT TTTTT | 2 |
| | TTTTT TTTTT TTTTT TTTTT TTTTT AATCAGCCATACCACATTTG | |
| DNA tag | /5Phos/AGAGA ATGGA TAGGT TGTGT TTTTT TTTTT TTTTT | 3 |
| | TTTTT TTTTT TTTTT TTTTT TTTTT AATCAGCCATACCACATTTG | |
| DNA tag | /5Phos/TGTGT TAGGT ATGGA AGAGA TT +TT +TT +TT +TT +TT | 4 |
| | +TT +TT +TT +TT +TT +TTT AATCAGCCATACCACATTTG | |
| DNA letter | TTTTTTTTTTTTTTTTTTTT/iSpPC/AGAGA | 5 |
| DNA letter | TTTTTTTTTTTTTTTTTTTT/iSpPC/ATGGA | 5 |
| DNA letter | TTTTTTTTTTTTTTTTTTTT/iSpPC/TAGGT | 5 |
| DNA letter | TTTTTTTTTTTTTTTTTTTT/iSpPC/TGTGT | 5 |
| DNA letter | GTAAAACGACGGCCAG/iSpPC/AGAGA | 6 |
| DNA letter | GTAAAACGACGGCCAG/iSpPC/ATGGA | 6 |
| DNA letter | GTAAAACGACGGCCAG/iSpPC/TAGGT | 6 |
| DNA letter | GTAAAACGACGGCCAG/iSpPC/TGTGT | 6 |
| PCR | GGATTATTCATACCGTCCCA | 7 |
| primer | | |
| PCR | CAAATGTGGTATGGCTGATT | 8 |
| primer | | |
| Splints for | ACCTATCCATTCTCT | 9 |
| letter | ACACATCCATTCTCT | 10 |
| | TCCATACCTATCTCT | 11 |
| | ACACAACCTATCTCT | 12 |
| | TCCATACACATCTCT | 13 |
| | ACCTAACACATCTCT | 14 |
| | ACCTATCTCTTCCAT | 15 |
| | ACACATCTCTTCCAT | 16 |
| | TCTCTACCTATCCAT | 17 |
| | ACACAACCTATCCAT | 18 |
| | TCTCTACACATCCAT | 19 |
| | ACCTAACACATCCAT | 20 |
| | TCCATTCTCTACCTA | 21 |
| | ACACATCTCTACCTA | 22 |
| | TCTCTTCCATACCTA | 23 |
| | ACACATCCATACCTA | 24 |
| | TCTCTACACAACCTA | 25 |
| | TCCATACACAACCTA | 26 |
| | TCCATTCTCTACACA | 27 |
| | ACCTATCTCTACACA | 28 |
| | TCTCTTCCATACACA | 29 |
| | ACCTATCCATACACA | 30 |
| | TCTCTACCTAACACA | 31 |
| | TCCATACCTAACACA | 32 |
| Splint for | TCC ATT CTC TTG GGA CGG TA | 33 |
| primer | ACC TAT CTC TTG GGA CGG TA | 34 |
| | ACA CAT CTC TTG GGA CGG TA | 35 |
| | TCT CTT CCA TTG GGA CGG TA | 36 |
| | ACC TAT CCA TTG GGA CGG TA | 37 |
| | ACA CAT CCA TTG GGA CGG TA | 38 |
| | TCT CTA CCT ATG GGA CGG TA | 39 |
| | TCC ATA CCT ATG GGA CGG TA | 40 |
| | ACA CAA CCT ATG GGA CGG TA | 41 |
| | TCT CTA CAC ATG GGA CGG TA | 42 |
| | TCC ATA CAC ATG GGA CGG TA | 43 |
| | ACC TAA CAC ATG GGA CGG TA | 44 |

### DETAILED DESCRIPTION

The present disclosure generally relates to systems and methods for spatially barcoding or identifying molecules in a sample. Certain embodiments are directed to multidimensional spatial omics tools with targeted or untargeted biomolecule profiling, highly parallelized barcoding, and sub-cellular resolution. Some aspects may introduce spatial barcodes to biomolecules using light and sequence them to decipher their content and position inside cells and blocks of tissue. The spatial barcoding can be achieved using light beams that can address pixels in a 3D space, e.g., without having any dead space between them. In certain cases, untargeted RNA measurement, 3D profiling of tissues, parallelized barcoding, no dead space, and/or the possibility of single-cell sequencing provide ways to collect molecular and spatial information from tissues, e.g., for discovery in cancer biology, immunology, neuroscience, and developmental biology. In certain cases, having an untargeted, fast, and/or 3D spatial omics technology like the ones described herein can facilitate discovery, e.g., for studying spatial organization of molecular features of tissues, organs, and organisms, etc., which may allow their study in healthy, diseased, drugged state, or the like.

Certain embodiments are generally directed to systems and methods of spatially controlling the attachment of DNA letters or sequences to molecules (for example, attachment to other nucleic acids such as DNA, RNA, etc., proteins, or the like) within a sample. In some cases, the molecules that the DNA letters are attached to may be tagged with a sequence such as a DNA tag, e.g., as described herein, which the DNA letters can be attached to. The sample may be a cell, tissue, or other suitable sample. The DNA tag or other nucleic acid sequence may be used in some cases to identify molecules, spatial positions, or other characteristics of the sample, e.g., by binding to specific molecules within the sample, e.g., covalently or noncovalently. In some cases, a plurality of DNA letters or sequences that are attached to a DNA tag may be used to form a barcode (e.g., comprising a plurality of DNA letters), and the positions of such DNA letters or sequences may be spatially controlled, for example, using the application of light, e.g., such that in locations where light is applied, the DNA letters or sequences can be added to the sample, e.g., to DNA tags, other nucleic acids, etc., attached to the sample, while in locations where such light is not applied, the DNA letters or sequences may not be added to such DNA tags, nucleic acids, etc. that may be present in those locations The light may be controllably applied in a variety of configurations, including 2 and 3 dimensional configurations, which may allow for spatial control in 2 or even 3 dimensions within a sample, e.g., of the positioning of such DNA letters or sequences within the sample, e.g., as barcodes. Accordingly, in certain embodiments, by repeated rounds of the application of DNA letters or sequences, and/or light exposure, molecules attached to DNA tags or other nucleic acids that are present at different locations in a sample may have different barcodes that have been attached there. Afterwards, such DNA tags and/or barcodes may be extracted (e.g., removed from the sample), and in some cases sequenced, for example, using commonly-available sequencing techniques to identify barcodes or other nucleic acids extracted from the sample. Because the barcodes may encode spatial information, the original location of the barcodes within the sample may be determined in certain embodiments by analyzing the sequences of the barcodes. In addition, the molecular identity may be inferred by the sequence of the DNA tag or barcode in some cases, e.g., based on the DNA tags originally added to the sample.

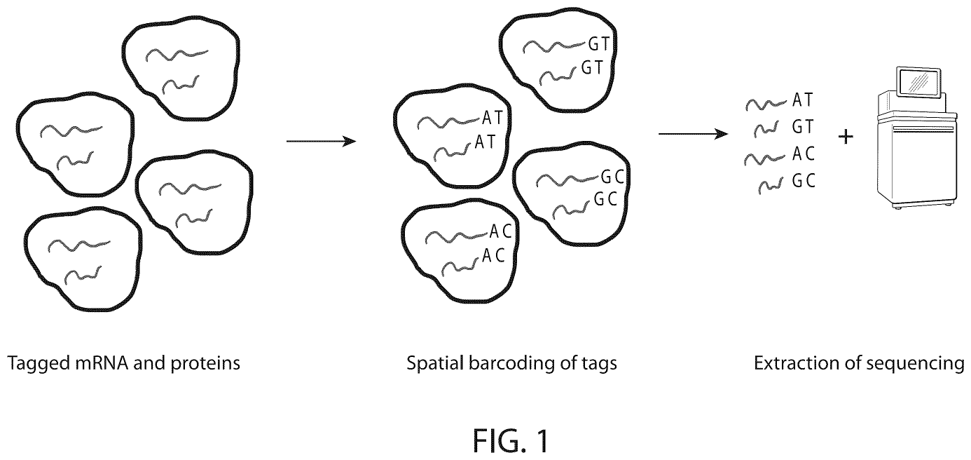

For example, in one set of embodiments, nucleic acids (e.g., comprising DNA and/or RNA), such as DNA letters, may be added to a sample in spatially controlled positions by the application of light. Thus, different spatial locations within a sample may have attached to them different nucleic acids, which may allow the spatial locations to be uniquely identified. In some cases, the application of light may be controlled such that at certain spatial positions, multiple nucleic acid sequences can be added, e.g., to the sample, and/or to other nucleic acids such as DNA tags, for example, that may be present within the sample. The other nucleic acids within the sample may be endogenous to the sample, and/or may have been previously attached, e.g., in prior rounds of nucleic acid attachments, using these or other techniques. In this way, in accordance with certain embodiments, a “barcode” of DNA letters or other nucleic acids can be formed, e.g., by using the application of light (for example, as beams of light) to control the addition of various nucleic acids at those spatial positions. In contrast, in other locations of the sample, the nucleic acids and/or specific combinations of nucleic acids may not present. Thus, in some embodiments, various spatial positions within the sample can be determined based on the nucleic acids that are present. One schematic illustration of this process is shown in the example of FIG. 1.

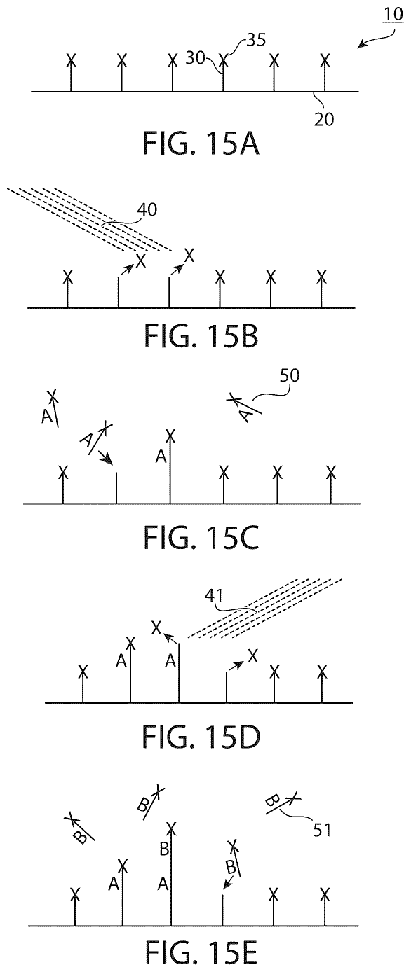

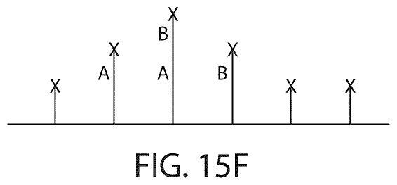

As another non-limiting example, in FIG. 15A, system 10 has a variety of nucleic acids 30 (e.g., DNA tags such as those described herein) attached to the sample 20 at various locations. The sample may be, for example, a cell, a tissue, or other suitable sample. At least some of the nucleic acids are blocked with blocking group 35, represented by an X in this figure. The blocking group may be photocleavable in some cases. Examples of blocking groups are discussed in more detail below, as well as in a patent application filed on even date herewith, entitled “Chemical Ligation Techniques,” by Zhuang, et al.

In FIG. 15B, light 40 is applied to a location of the sample. In particular, it should be noted that the light is directed at specific locations, while other locations within sample 20 do not receive light 40 (or they may receive some incidental light, but the light is not specifically directed at those locations). In locations where light 40 is directed, the light may be able to cause the removal of the blocking groups, for instance, as discussed herein. However, in other locations in the sample, the blocking groups are not removed.

In FIG. 15C, nucleic acids 50 are added, comprising a sequence A and a blocking group (represented by an X). Nucleic acids 50 can be added to the nucleic acids in the sample, except when blocked by a blocking group. Accordingly, in locations where the light in FIG. 15B was directed, sequence A may be added to the nucleic acid. However, in other locations, due to the blocking group, sequence A cannot be added. Afterwards, unreacted nucleic acids 50 may be washed away or otherwise removed from the sample.

This process may be repeated any number of times, in various embodiments; only two rounds are discussed here for ease of understanding. However, there may be 3, 4, 5, 7, 10, 15, 20, or any other suitable number of rounds. In FIG. 15D, second light 41 is applied to the sample. Light 41 may be directed to any location in the sample, and may be used in some cases to remove blocking groups in locations where the light is directed. It should be noted that in this figure, some locations of light 41 overlap with locations that were previously exposed to light 40 in FIG. 15B. However, other locations of sample 20 are exposed to light 41, but were not previously exposed to light 40 in FIG. 15B. It should also be understood that this is for illustrative purposes only; in other embodiments, for example, light 40 and light 41 may be completely overlapping, may not overlap at all, or may overlap in specific locations as was shown in FIG. 15D, etc.

In FIG. 15E, nucleic acids 51 are added, comprising a sequence B and a blocking group (represented by an X). Nucleic acids 51 can be added to the nucleic acids in the sample, except when blocked by a blocking group. Accordingly, in locations where the light in FIG. 15D was directed, sequence B may be added to the nucleic acid. However, in other locations, due to the blocking group, sequence B may not be added to sample 20.

It should be noted that, as shown schematically in FIG. 15E, some parts of light 41 were applied to locations that had previously been exposed to light 40 and nucleic acids 50 (represented by sequence A), and thus, in those locations, sequence B can be attached to sequence A. However, in other locations not previously exposed to light 40, no sequence A is present, and those nucleic acids may contain only sequence B.

In some embodiments, after the attachment of various nucleic acids (which may each include, for example, 1, 2, 3, 4, 5, or any suitable number of nucleotides, e.g., as discussed herein) such nucleic acids can be removed from the sample, e.g., to be sequenced, or for other purposes. It should be noted that, in this example, locations which were exposed to both light 40 and light 41 can be spatially identified by the presence of both sequence A and sequence B in the same nucleic acid, even if the nucleic acid is subsequently removed or extracted from the sample. In contrast, locations that were not exposed to both light 40 and light 41 may contain only sequence A, only sequence B, or neither A nor B. Accordingly, based on the nucleic acid sequences that are present, e.g., as determined by sequencing or other suitable techniques, the spatial positions of the nucleic acids within the sample can be determined, for example, based on the spatial information encoded within those nucleic acids, even if the nucleic acids have been removed or extracted from the sample.

As mentioned, in some embodiments, some nucleic acids (e.g., in a DNA tag, DNA letter, or the like) may include one or more blocking groups which may inhibit additional nucleic acids from being added to those nucleic acids. However, such blocking groups can be removed under certain conditions, for example, upon being exposed to light, and accordingly, after suitable exposure, one or more nucleic acids can be added to them. In addition, in certain cases, the nucleic acids that are added may also contain blocking groups, and/or a blocking group may be attached to the added nucleic acids, e.g., such that afterwards, the blocking groups are able to inhibit further additional nucleic acids from being added (for example, until the blocking group is exposed to light, etc.). By repeating this process, in various embodiments, any suitable number of nucleic acids may be arbitrarily added to any desired spatial positions within the sample.

For example, in one embodiment, a nucleic acid may include an initial sequence, a blocking group comprising a photocleavable linker and a spacer sequence, e.g., connecting the spacer sequence to the initial sequence. The application of light (e.g., ultraviolet light) may cause the cleavage of the photocleavable linker, e.g., such that the spacer sequence can be removed from the nucleic acid (e.g., via diffusion). In some cases, the cleavage may cause the exposure of a 5′ phosphate group. After removal of the blocking group, additional nucleic acids may be attached to the initial nucleic acid, e.g., ligated to the initial nucleic acid (for example, using a DNA-binding enzyme, such as DNA polymerase) using the 5′ phosphate. Further examples of such blocking groups, and methods of using them, may be seen in a patent application filed on even date herewith, entitled “Chemical Ligation Techniques,” by Zhuang, et al.

As non-limiting examples, certain aspects of the present disclosure are now described. In one set of embodiments, a DNA tag (or other nucleic acid) can be attached to RNA and/or proteins inside a cell, tissue, or other suitable sample. The DNA tag may also be attached to other targets in a sample in certain cases. The DNA tag may be used to identify certain targets within the sample. For example, as discussed herein, in some cases, the DNA tag (or other nucleic acid) may contain a moiety able to recognize a specific target within the sample (e.g., a protein, nucleic acid, or the like that may be present within the sample), and the DNA tag can be bound to the sample (e.g., covalently or noncovalently), and used as an attachment point for subsequent nucleic acids (e.g., DNA letters), which can then be sequenced or identified. In some cases, the attachment of subsequent nucleic acids to such DNA tags or other nucleic acids within the sample can be controlled, e.g., spatially, and in some cases, various nucleic acids may be attached and used to encode the spatial locations of the DNA tags or other nucleic acids within the sample.

A sample may include a cell culture, a suspension of cells, a biological tissue, a biopsy, an organism, or the like. The sample can also be cell-free but nevertheless contain nucleic acids in some cases. If the sample contains a cell, the cell may be a human cell, or any other suitable cell, e.g., a mammalian cell, a fish cell, an insect cell, a plant cell, or the like. More than one cell may be present in some cases. In some embodiments, cells, tissues, or other samples may be permeabilized, e.g., to allow such fluid flow to occur. In certain embodiments, components within a cell, tissue, or other sample may be fixed, e.g., prior to exposure to DNA tags. Those of ordinary skill in the art will be familiar with techniques for permeabilizing or fixing cells or other samples.

The DNA tags attached to the sample can be used for a variety of purposes. For example, the DNA tags can be used as attachment points for the subsequent of DNA letters or other nucleic acids, e.g., in a spatially controlled manner. In some cases, such nucleic acids can be used to extract or otherwise determine RNA and/or protein content, and/or spatial position information of the sample, e.g., as discussed below. In addition, the DNA tags may be extended in certain cases by reverse transcription to copy RNA information onto it. The RNA may be mRNA, miRNA, siRNA, and/or other RNAs and/or other nucleic acids present in a cell, tissue, or other suitable sample. In some cases, position-dependent barcodes or other suitable nucleic acids can be introduced and added to the DNA tags (or other nucleic acids) in the sample, e.g., as discussed herein. For example, a barcode may be formed on a DNA tag by the addition for DNA letters or other nucleic acids. This can be achieved, for example, through in-situ DNA synthesis, where the DNA synthesis may be controlled using spatial light patterning. Multiple rounds of attachment of DNA letters or other nucleic acids may be used in some embodiments, e.g., to form the barcodes. Afterwards, the DNA tags or barcodes can be removed or extracted from the sample, and can be sequenced to obtain information, for example, about the content and/or spatial information of molecules within the sample. Examples of these are discussed in detail below.

In one set of embodiments, DNA tags, or other nucleic acids, may be introduced to a sample. The DNA tags or other nucleic acids may, in certain embodiments, be used as an attachment point to attach subsequent DNA letters or other nucleic acids, such as is described herein. In some cases, the DNA tags, or other nucleic acids, may be attached to or immobilized to the sample, e.g., covalently or noncovalently, and used for subsequent analysis. In some embodiments, a DNA tag or other nucleic acid may include an antibody, a nucleic acid sequence, or the like that is able to recognize specific features within a sample. The DNA tag or other nucleic acid may be immobilized with respect to those features (e.g., due to covalent or noncovalent binding, etc.), and accordingly those features can be identified, e.g., spatially within the sample, as is described herein. Those of ordinary skill in the art will be aware of methods and systems for attaching or conjugating nucleic acids such as DNA to proteins such as antibodies using a DBCO-Azide reaction, maleamide-NHS ester reaction or the like.

For example, in certain embodiments, as discussed herein, the DNA tags, or other nucleic acids, may be attached to any desired biomolecules that are suspected of being present within a sample. For example, the DNA tags may be attached to RNA within a sample. The RNA may be coding and/or non-coding RNA. For example, the RNA may encode a protein. Non-limiting examples of RNA that may be studied include mRNA, siRNA, rRNA, miRNA, tRNA, lncRNA, snoRNAs, snRNAs, exRNAs, piRNAs, viral RNA, or the like. As another example, the DNA tags, or other nucleic acids, may be attached to DNA within a sample. The DNA may include chromosome DNA, mitochondria DNA, chloroplast DNA, plasmid DNA, or the like. DNA fragments (e.g., from a virus) may also be studied in some cases. As yet another example, the DNA tags, or other nucleic acids, may be attached to proteins in a sample.

A variety of techniques for attaching a DNA tag or other nucleic acids to a sample are known to those of ordinary skill in the art. For example, an antibody to a protein suspected of being in a sample may be used, to which a DNA tag may be attached to. Other techniques for attaching a DNA tag to a molecule within a sample will be known to those of ordinary skill in the art, and can be used in still other embodiments. In addition, it should be understood that more than one type of molecule may be studied in a sample in certain cases.

As a non-limiting example of determining spatial position of mRNA, DNA tags comprising of a poly-T may be attached to the sample. The DNA tags comprising poly-T may be extended, for example, by reverse transcription using a reverse transcriptase enzyme, which may be used to copy the endogenous mRNA information onto the DNA tag. The DNA tag may have a photocleavable blocking group. In some cases, a nucleic acid comprising a photocleavable blocking group may be attached to the DNA tag (or other nucleic acid) using methods of DNA synthesis or ligation described herein, as well as in a patent application filed on even date herewith, entitled “Chemical Ligation Techniques,” by Zhuang, et al. As described herein, DNA letters may then be added in certain embodiments using spatial light patterning, e.g., to build up a spatial barcode. The DNA tags with barcodes attached to mRNA may be extracted, for example, using an RNAse enzyme. The extracted nucleic acids may be sequenced to determine the sequence of mRNA and/or the DNA tag, along with its spatial location. Various processes of determining the mRNA sequence from the DNA tag will be known to those of ordinary skill in the art. In some cases, the mRNA identities along with spatial information of a sample may be determined.

As a non-limiting example of determining spatial position proteins in a sample, DNA tags or other nucleic acids, e.g., comprising an oligonucleotide conjugated antibody able to specifically recognize a protein of interest may be attached to a sample. In some embodiments, the oligonucleotide conjugated antibody may be of a predetermined sequence, e.g., to allow proteins to be distinguished from another. In some cases, a variety of proteins may be distinguished, e.g., at least 1, at least 2, at least 3, at least 5, at least 10, at least 20, at least 30, at least 50, at least 100, at least 200, at least 300, at least 400, at least 500, at least 600, at least 700, at least 800, at least 900, or at least 1,000 proteins may be distinguished.

The DNA tag may have a photocleavable blocking group in certain embodiments. In some cases, a nucleic acid comprising a photocleavable blocking group may be attached to a DNA tag or other nucleic acid, for instance, using methods of DNA synthesis or ligation described herein, as well as in a patent application filed on even date herewith, entitled “Chemical Ligation Techniques,” by Zhuang, et al. As described herein, DNA letters may in some cases be added using spatial light patterning to build up a spatial barcode. The DNA tags with barcodes attached to the protein may be extracted, for example, using a proteinase enzyme. The extracted nucleic acids may be sequenced to determine the sequence of the DNA tag along with the barcodes. By filtering for sequences corresponding to known oligonucleotide sequences conjugated to antibodies, the spatial location of the corresponding protein may be determined in some embodiments.

As a non-limiting example of determining DNA (which may be endogenous or introduced, for example, using a lentivirus) in a sample, a plurality of DNA tags containing complementary sequences to the DNA of interest may be attached to the sample. If the DNA is double stranded, the DNA may be denatured in some cases. Methods of denaturing, for example, using formamide, will be known to those of ordinary skill in the art. In addition, the DNA tag or other nucleic acids may be bound to biotin in certain embodiments. In some cases, DNA letters or other nucleic acids may be added, for instance, using spatial light patterning to build up a barcode. The sample may, in some cases, be heat denatured and DNA tags or other nucleic acids with barcodes may be extracted, for example, by using streptavidin to precipitate out the DNA tags containing biotin. The extracted DNA tags with barcodes may be sequenced, e.g., as discussed herein. The sequence of the DNA tag may be used to identify the target DNA. In some cases, the sequence of the barcode formed by the DNA letters or other nucleic acids may be used to identify the location of the target DNA within a sample.

In some embodiments, mRNA, proteins, DNA, and/or other targets can be studied together in the same sample, e.g., using the appropriate combination of DNA tags.

The DNA tag, or other nucleic acid, may have any suitable length. For example, the DNA tag or other nucleic acid may have a length of at least 1, at least 2, at least 3, at least 4, at least 5, at least 6, at least 7, at least 8, at least 9, at least 10, at least 12, at least 15, at least 20, at least 25, at least 30, at least 35, at least 40, at least 50, at least 60, at least 65, at least 70, at least 75, at least 100, at least 200, at least 300, at least 400, at least 500, at least 600, at least 700, at least 800, at least 900, or at least 1,000 nucleotides in length. In addition, the DNA tag or other nucleic acid may have a length of no more than 1,000, no more than 900, no more than 800, no more than 700, no more than 600, no more than 500, no more than 400, no more than 300, no more than 200, no more than 100, no more than 75, no more than 70, no more than 65, no more than 60, no more than 50, no more than 40, no more than 35, no more than 30, no more than 25, no more than 20, no more than 15, no more than 12, no more than 10, no more than 9, no more than 8, no more than 7, no more than 6, no more than 5, no more than 4, no more than 3, or no more than 2 nucleotides. Combinations of any of these are also possible. For example, the DNA tag or other nucleic acid may have between 2 and 6 nucleotides, between 15 and 35 nucleotides, between 20 and 50 nucleotides, between 70 and 100 nucleotides, etc.

In addition, in certain cases, some or all of the DNA tags, or other nucleic acids, may include a blocking agent, e.g., that inhibits the attachment of additional nucleic acids. Non-limiting examples of blocking agents are discussed in more detail herein, as well as in a patent application filed on even date herewith, entitled “Chemical Ligation Techniques,” by Zhuang, et al.

In various embodiments, DNA tags or other nucleic acids may be used that can recognize any of a wide variety of targets that may be present within a sample. The targets may include, for example, nucleic acids, proteins, enzymes, antibodies, receptors, complementary nucleic acid strands, aptamers, or the like, or targets may include targets that are linked to such nucleic acids, proteins, etc. The determination of targets, such as nucleic acids within the cell or other sample, may be qualitative and/or quantitative. In addition, the determination may also be spatial in certain embodiments, e.g., the position of the nucleic acids, or other targets, within the cell or other sample may be determined, for example, in two or three dimensions such as discussed herein. In some embodiments, the positions, number, and/or concentrations of nucleic acids, or other targets, within the cell or other sample may be determined.

In addition, in some embodiments, a significant portion of the nucleic acids within the cell or other sample may be studied. In some cases, for example, enough of the RNA present within a cell (or other sample) may be determined so as to produce a partial or complete transcriptome of the cell or other sample. In some cases, at least 1, at least 2, at least 3, at least 4, at least 7, at least 8, at least 12, at least 14, at least 15, at least 16, at least 20, at least 22, at least 30, at least 31, at least 32, at least 50, at least 63, at least 64, at least 72, at least 75, at least 100, at least 127, at least 128, at least 140, at least 255, at least 256, at least 500, at least 1,000, at least 1,500, at least 2,000, at least 2,500, at least 3,000, at least 4,000, at least 5,000, at least 7,500, at least 10,000, at least 12,000, at least 15,000, at least 20,000, at least 25,000, at least 30,000, at least 40,000, at least 50,000, at least 75,000, or at least 100,000 types of RNAs may be determined within a cell or other sample.

In some cases, the transcriptome of a cell may be determined. It should be understood that the transcriptome generally encompasses all RNA molecules produced within a cell, not just mRNA. Thus, for instance, the transcriptome may also include rRNA, tRNA, siRNA, etc. in certain instances. In some embodiments, at least about 0.01%, at least about 0.1%, at least about 1%, at least 5%, at least 10%, at least 15%, at least 20%, at least 25%, at least 30%, at least 40%, at least 50%, at least 60%, at least 70%, at least 80%, at least 90%, or 100% of the transcriptome of a cell may be determined.

Furthermore, in some embodiments, targets to be determined can include various targets that are linked to nucleic acids, proteins, or the like. For instance, in one set of embodiments, a binding entity able to recognize a target may be conjugated to a nucleic acid probe. The binding entity may be any entity that can recognize a target, e.g., specifically or non-specifically. Non-limiting examples include enzymes, antibodies, receptors, complementary nucleic acid strands, aptamers, or the like. For example, an oligonucleotide-linked antibody may be used to determine a target. The target may bind to the oligonucleotide-linked antibody, and the oligonucleotides determined as discussed herein.

As a non-limiting example, to identify proteins, the proteins within a sample may be tagged with an antibody conjugated to DNA oligonucleotide that has a specific DNA sequence for each protein type. A complementary DNA oligonucleotide may be used as a DNA tag, for example, and in some cases, the DNA tag may be selected to be able to identify the protein, e.g., during subsequent sequencing.

In addition, certain aspects as discussed herein are generally directed to systems and methods for controlling the attachment of DNA “letters” or nucleic acids sequences to molecules such as DNA tags or other nucleic acids that may be present within a sample. As discussed herein, the placement of such DNA letters or other nucleic acids may be spatially controlled, e.g., by using the application of light, such that in locations where light is applied, the DNA letters or other nucleic acids are added to DNA tags or other nucleic acids within the sample, while in locations where light is not applied (or where the light is not specifically directed at those locations), the DNA letters or other nucleic acids may not be added to such DNA tags, nucleic acids, etc., within the sample. Thus, by controlling where light is applied to a sample, the locations where nucleic acids are added can be controlled, e.g., spatially. Thus, different nucleic acid sequences can be formed or synthesized at different spatial locations within a sample.

In some embodiments, this may be advantageously used to create unique nucleic acid sequences or “barcodes” that encode certain types of information, such as their spatial location. In addition, in certain embodiments, such spatial location information may be used to create a 2- or even 3-dimensional image about the sample. In certain embodiments, the barcodes may include error-detecting and/or error-correcting codes, or other types of information, e.g., in addition to and/or instead of spatial location information.

The DNA “letters” or other nucleic acid sequences may be of any length. If more than one DNA letter is present, the DNA letters may each independently have the same or different lengths. A DNA letter may be thought of as encoding a unit of information. In some embodiments, multiple DNA letters may be combined together to form a barcode, e.g., that encodes information, much as a word may be composed of multiple letters.

Thus, if more than one DNA letter or other nucleic acids is used, e.g., to form a barcode, the DNA letters or other nucleic acids that are used may each independently have the same or different lengths. For instance, the DNA letter or other nucleic acid may be at least 1, at least 2, at least 3, at least 4, at least 5, at least 6, at least 7, at least 8, at least 9, at least 10, at least 12, at least 15, at least 20, at least 25, at least 30, at least 35, at least 40, at least 50, at least 60, at least 65, at least 70, at least 75, at least 100, at least 200, at least 300, at least 400, at least 500, at least 600, at least 700, at least 800, at least 900, or at least 1,000 nucleotides in length. In some cases, the DNA letter or other nucleic acid may be no more than 1,000, no more than 900, no more than 800, no more than 700, no more than 600, no more than 500, no more than 400, no more than 300, no more than 200, no more than 100, no more than 75, no more than 70, no more than 65, no more than 60, no more than 50, no more than 40, no more than 35, no more than 30, no more than 25, no more than 20, no more than 15, no more than 12, no more than 10, no more than 9, no more than 8, no more than 7, no more than 6, no more than 5, no more than 4, no more than 3, or no more than 2 nucleotides in length. Combinations of any of these are also possible, e.g., a DNA letter may have a length of between 10 and 30 nucleotides, between 5 and 8 nucleotides, between 5 and 50 nucleotides, between 10 and 20 nucleotides, between 4 and 9 nucleotides, between 10 and 30 nucleotides, between 300 and 500 nucleotides, etc.

In addition, a population of barcodes may have any suitable number of DNA letters or nucleic acid sequences that defines the population of barcodes. For example, a population of barcodes may use 1, 2, 3, 4, 5, 6, 7, 8, 9, 10, 11, 12, 13, 14, 15, 16, 17, 18, 19, 20, etc. DNA letters or nucleic acid sequences. More than 20 are also possible in some embodiments. In addition, in some embodiments, the population of barcodes maybe formed from at least 1, at least 2, at least 3, at least 4, at least 5, at least 6, at least 7, at least 8, at least 9, at least 10, at least 11, at least 12, at least 13, at least 14, at least 15, at least 16, at least 20, at least 24, at least 32, at least 40, at least 50, at least 60, at least 64, at least 80, at least 100 DNA letters or nucleic acid sequences. In certain cases, no more than 100, no more than 80, no more than 64, no more than 60, no more than 50, no more than 40, no more than 32, no more than 24, no more than 20, no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than one DNA letters or nucleic acid sequences may be present in a population of barcodes. Combinations of any of these are also possible, e.g., a population of barcodes may comprise between 10 and 15, between 7 and 10, between 60 and 100, between 20 and 40, etc. DNA letters or nucleic acid sequences in total. Additionally, the barcodes in a population of barcodes may each independently have the same or different numbers of DNA letters or other nucleic acid sequences.

As a non-limiting example of an approach to combinatorically identifying a relatively large number of barcodes from a relatively small number of DNA letters (or other nucleic acid sequences), a population of 4 different DNA letters is now described. It should be understood that although 4 DNA letters are used in this example for ease of explanation, in other embodiments, larger numbers of barcodes may be realized, such as discussed herein, for example, by using 5, 8, 10, 16, 32, etc. or more different DNA letters, or any other suitable number of DNA letters such as is described herein, depending on the application.

As a non-limiting example, the DNA letters may be chosen as the set of 4 bases: A, T, G, and C (or in some cases, a subset of these, and/or other bases, e.g., non-naturally occurring bases). The first letter of the barcode can be any one of the four bases. The second letter of the barcode can also be any one of the four bases, e.g., to create up to 4² possibilities (AA, AT, AG, AC, TA, TT, TG, TC, GA, GT, GG, GC, CA, CT, CG, CC). The third letter of the barcode can also be any one of the four bases, e.g., to create up to 4³ possibilities. For m letters, there could be up to 4^{m} possibilities. In some embodiments, the total number of possibilities may be chosen to be greater than the number of unique locations to be barcoded. So, therefore, in this example, for N unique locations, there could be at least m~log₄(N) bases. For example, a sample block of 5 mm×5 mm×500 μm with a subcellular resolution of 3 um may contain 4.8×10⁸ voxels and can be represented by as low as log₄(4.8×10⁸) bases. Accordingly, as discussed herein, since DNA sequencing may be used in some embodiments to preserve the order that the DNA tags are added, even a relatively large number of unique barcodes may be obtained from a relatively small number of DNA letters in certain embodiments.

As a non-limiting example, if each of the barcodes contain two different DNA letters, then by using 4 such DNA letters (A, B, C, and D), up to 6 barcodes may be used if ordering is not essential and repeats are forbidden (AB, AC, AD, BC, BD, CD), or up to 16 if ordering is essential and repeats are required (AA, AB, AC, AD, BA, BB, BC, BD, CA, CB, CC, CD, DA, DB, DC, DD). This can be increased even further if the barcodes need not all contain the same number of DNA letters; for example, up to 20 could be obtained in this illustrative example (A, AA, AB, AC, AD, B, BA, BB, BC, BD, C, CA, CB, CC, CD, D, DA, DB, DC, DD).

As another non-limiting example of using letters with a combination of bases, the DNA letters are among these 6 different 5 base long sequences: AGAGA, ATGGA, TAGGT, TGTGT, AAGGT, TTGGA. A barcode of one DNA letter can have one of the six possibilities. A barcode with two DNA letters can have 6 options for the first letter and 6 options for the second letter for a total of 6² total possibilities, and can be used to produce sequences such as AGAGA AGAGA (SEQ ID NO: 45) or AAGGT TTGGA (SEQ ID NO: 46), etc. For a barcode with three DNA letters, there can be up to 63 possibilities, for example, with sequences that look like AGAGA TGTGT AGAGA (SEQ ID NO: 47) or TGTGT TGTGT TTGGA (SEQ ID NO: 48), etc. For a barcode with m DNA letters, there would be 6^{m} possibilities, which grows exponentially with number of DNA letters.

As another non-limiting example of using letters with a combination of bases, the DNA letters are among these 6 different 5 base long sequences: AGAGA, ATGGA, TAGGT, TGTGT, AAGGT, TTGGA. In this example, in addition, a DNA letter may be chose to not be repeated with the previous two DNA letters. A barcode of one DNA letter can have one of the six possibilities. A barcode with two DNA letters can have 6×5 possibilities, since there cannot be a repeat with the previous DNA letter, and this can be used to produce sequences such as AGAGA ATGGA (SEQ ID NO: 49) or TAGGT TTGGA (SEQ ID NO: 50), etc. For a barcode with three DNA letters, there can be up to 6×5×4 possibilities, e.g., with sequences such as AGAGA TGTGT ATGGA (SEQ ID NO: 51) or TGTGT ATGGA TTGGA (SEQ ID NO: 52), etc. For a barcode with four DNA letters, there can be up to 6×5×4² possibilities, with sequences such as AGAGA TGTGT ATGGA AGAGA (SEQ ID NO: 53). In some cases, some of the DNA letters may repeat and not be the same as the previous two DNA letters. As the barcode is expanded to m DNA letters, there would be up to 6×5×4^{(m-2)} possibilities, which may grow exponentially with number of DNA letters.

It should be understood that although the above examples used only 4 single-nucleotide DNA letters, for ease of understanding, in other embodiments, DNA “letters” or other sequences with more than one nucleotide can be used as well, which may be combined to form barcodes, e.g., of any suitable length. For example, a barcode may comprise at least 1, at least 2, at least 3, at least 4, at least 5, at least 6, at least 7, at least 8, at least 9, at least 10, at least 12, at least 15, at least 20, at least 25, at least 30, at least 35, at least 40, at least 50, at least 60, at least 65, at least 70, at least 75, or at least 100 DNA letters (or other sequences).

As but one non-limiting example, in one embodiment, 6 DNA “letters” may be defined as a=AG, b=GC, c=CA, d=TA, e=GT, and f=TC, and barcodes can be formed using these DNA letters, e.g., barcodes such as ab (AGGC), ac (AGCA), aab (AGAGGC), acd (AGCATA), etc. More complex barcodes can also be obtained in certain cases by using 2, 3, 4, 5, or more DNA “letters” or other sequences.

As these non-limiting examples illustrate, a relatively large number of barcodes may be formed or synthesized within a sample in certain embodiments, e.g., using techniques such as those described herein, based on a relatively small number of DNA letters or other nucleic acid sequences. Such barcodes may be used, for instance, to encode certain types of information, such as their spatial location, and/or other types of information. For example, a barcode may encode information about the experiment number, the applied experimental conditions, reaction conditions used, or the like. In addition in some cases, a barcode may encode more than one type of information, for example, spatial location and experiment number.

Thus, certain aspects are generally directed to systems and methods for encoding spatial information, e.g., within a barcode. For example, in one set of embodiments, various beams of light may be applied to specific locations of a sample to attach nucleic acids to the sample within those locations. By controlling where the light is applied, and thus where nucleic acids are attached to the sample, different locations within a sample may have different nucleic acids that are attached. Accordingly, the different nucleic acids may encode information about their spatial positions within the sample.

Accordingly, in certain embodiments, spatial information may be introduced with the barcodes. For example, in some embodiments, spatial locations within a sample may be discretized into pixels. The discretization of space may occur in 2 dimensions, or 3 dimensions in some instances, e.g., as discussed below. In addition, in certain embodiments, some or all spatial locations (e.g. pixel or voxel) may be identified, in some cases uniquely, with a specific sequence or barcode. As discussed below, within a spatial location, different barcodes may be defined. In some cases, such barcodes may be formed or synthesized, e.g., by attaching appropriate DNA letters or other nucleic acids to molecules such as DNA tags or other nucleic acids within that spatial location (e.g., if conditions are appropriate), for example, by controlling light such that it is applied to only those spatial locations to which appending or attachment of nucleic acids is desired. In some embodiments, a unique DNA letter or other nucleic acid sequence may be assigned to each spatial location, although in other embodiments, different spatial locations may be identified, for example, by using unique combinations of DNA letters, e.g., to form different barcodes identifying the different spatial locations. In various embodiments, DNA barcodes may be defined as various permutations or combinations of DNA letters or other nucleic acids, e.g., where the order matters or does not matter.

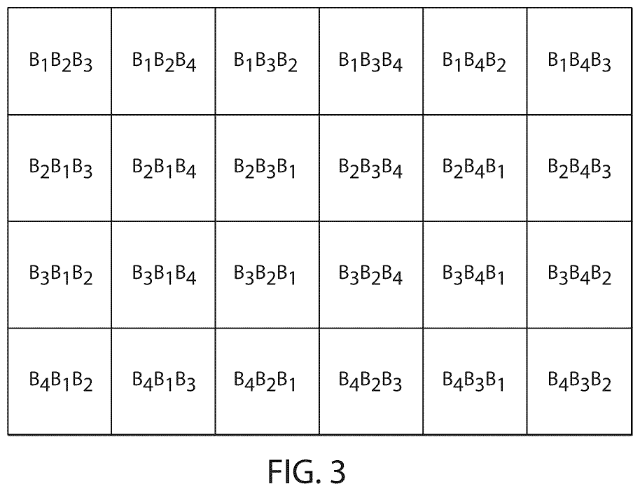

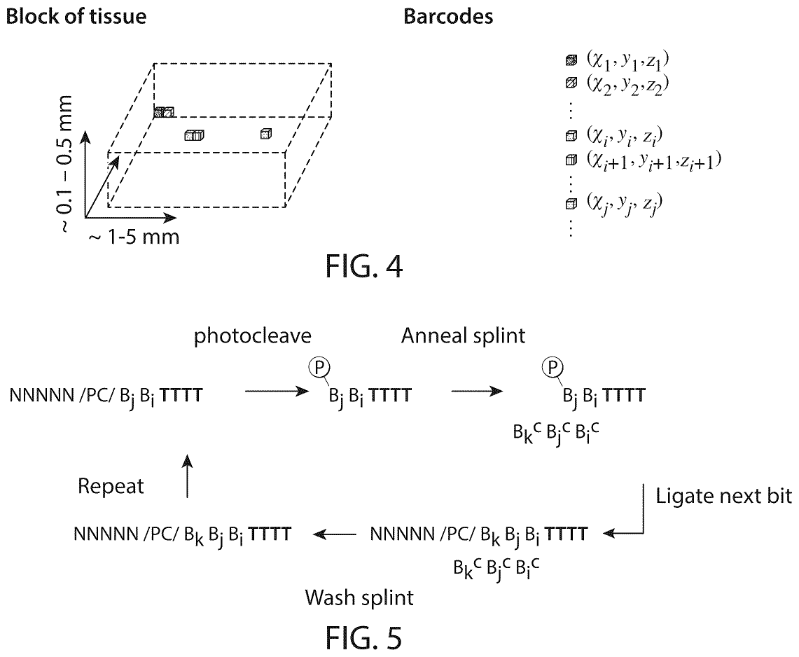

As a schematic non-limiting example, in FIG. 3, an example 2-dimensional region of space has been discretized, and each “pixel” uniquely identified with a barcode. The various possible DNA letters are represented as B₁, B₂, . . . , Bₘ and can include any suitable combination of bases, such as the four A, T, G, and C bases, and/or other non-naturally occurring bases in some cases. In addition, in some embodiments, some or all of the DNA letters may include more than one nucleotide, e.g., such as described herein. The DNA letters can be combined to form a barcode, for example, B₁B₂B₃, B₁B₂B₄, B₂B₁B₃, etc., as is shown in FIG. 4 with various combinations of B₁, B₂, B₃, and B₄ forming different barcodes. Other encoding techniques can also be used in other embodiments. For example, in some embodiments, the ordering may be important (e.g., in FIG. 3, B₁B₂B₃ and B₂B₁B₃ encode different spatial positions), while in other embodiments, ordering may not be important, and barcodes differentiated on the basis of content or concentrations and/or types of DNA letters or other nucleic acid sequences (as a non-limiting example, barcodes such as B₁B₂B₃ and B₂B₁B₃ may encode the same spatial position, while barcodes such as B₁B₁B₂ and B₁B₂B₂ may encode different spatial positions, e.g., due to the different concentrations of B's in each).

In some embodiments, various systems may allow for multiplex positional encoding in some cases, e.g., where unique combinations of DNA letters in a barcode may allow for different spatial positions to be uniquely identified, rather than using a unique DNA letter for each spatial location in other embodiments (however, in other embodiments, unique DNA letters may be used for each spatial location). Moreover, in some embodiments, the DNA letters may or may not be repeated within a barcode, e.g., in various applications.

In addition, certain aspects are generally directed to systems and methods for introducing DNA letters or other nucleic acids to a sample, e.g., at specific spatial locations within the sample. These may be attached to DNA tags or other nucleic acids contained within the sample, and/or to other targets in certain cases. A variety of reactions may be used to attach DNA letters or other nucleic acids to a sample. Non-limiting examples include those disclosed in a patent application filed on even date herewith, entitled “Chemical Ligation Techniques,” by Zhuang, et al.

In various embodiments, for example, methods of attaching DNA letters or other nucleic acids to a sample (e.g., to DNA tags or other nucleic acids such as other DNA letters) in a position-dependent manner can include in-situ light-directed enzymatic DNA synthesis or DNA ligation. In-situ synthesis or ligation may allow for the creation of barcodes in some cases, such as those discussed above, and control of light position in some embodiments may allow synthesis or ligation to occur, e.g., with spatial selectivity. The barcoding can be carried out over one or more rounds, with each round adding one DNA letter or other nucleic acid of the barcode, e.g., at spatially desired locations as controlled by the applied light.

As a schematic non-limiting example, to administer the first DNA letter or other nucleic acid (e.g., “B₁”), all locations that are encoded with B₁ as the first DNA tag (or other nucleic acid) can be illuminated with light in order to add that DNA tag (or other nucleic acid) to the sample. This can be repeated for locations that use B₂ as the first DNA tag, and so on until Bₘ. This completes the synthesis of the first DNA letter (or other nucleic acid) onto desired molecules within the sample, and this process can then be repeated to add the second DNA letter to the barcode, etc. until as many DNA letters are added as desired (for example, for the example shown in FIG. 3, three such rounds would be used since each barcode is formed from 3 unique DNA letters). After the DNA letters are added, the barcode that they represent may uniquely map onto a physical position on the sample (e.g., as illustrated in the example shown in FIG. 4) In some cases, the same DNA letter may be applied in different rounds (e.g., when the same DNA letter is used in different positions within the barcode), although in other cases, the same DNA letter may not necessarily be applied in different rounds (e.g., when different DNA letter are used in different positions within the barcode). Examples of methods for the light-directed enzymatic synthesis and ligation steps, followed by the description of creating spatial light patterns, are discussed in more detail herein.

In some embodiments, a first DNA letter or other nucleic acid may be added by using reactions which are controlled by light. The light may be applied to a sample, e.g., to one or more locations of the sample. In some cases, beams of light may be directed or focused onto specific locations of a sample, while other locations of the sample are not exposed to such beams of light. Instead, such locations may be left in the dark, or at least be illuminated only by incidental light, e.g., light not specifically directed at those locations. There may be one or more than one beam of light directed at a sample at specific points in time.

For example, in one set of embodiments, the beams of light may be used to remove a blocking group from a DNA tag or other nucleic acid, which may allow the DNA tag or other nucleic acid, which may allow additional nucleic acids to attach to it, e.g., via a ligation or other suitable reaction. The blocking group, when present, may inhibit the attachment of nucleic acids using DNA-binding enzymes, for example, DNA polymerases (e.g., TdT), DNA ligases (e.g., T4 DNA ligase), or the like. Other examples of DNA ligases include DNA ligase I, II, III, or IV, E. coli DNA ligase, etc.

In some cases, the blocking group may be attached via a photocleavable linker. Many such photocleavable linkers are commercially available. Non-limiting examples of photocleavable linkers are shown below:

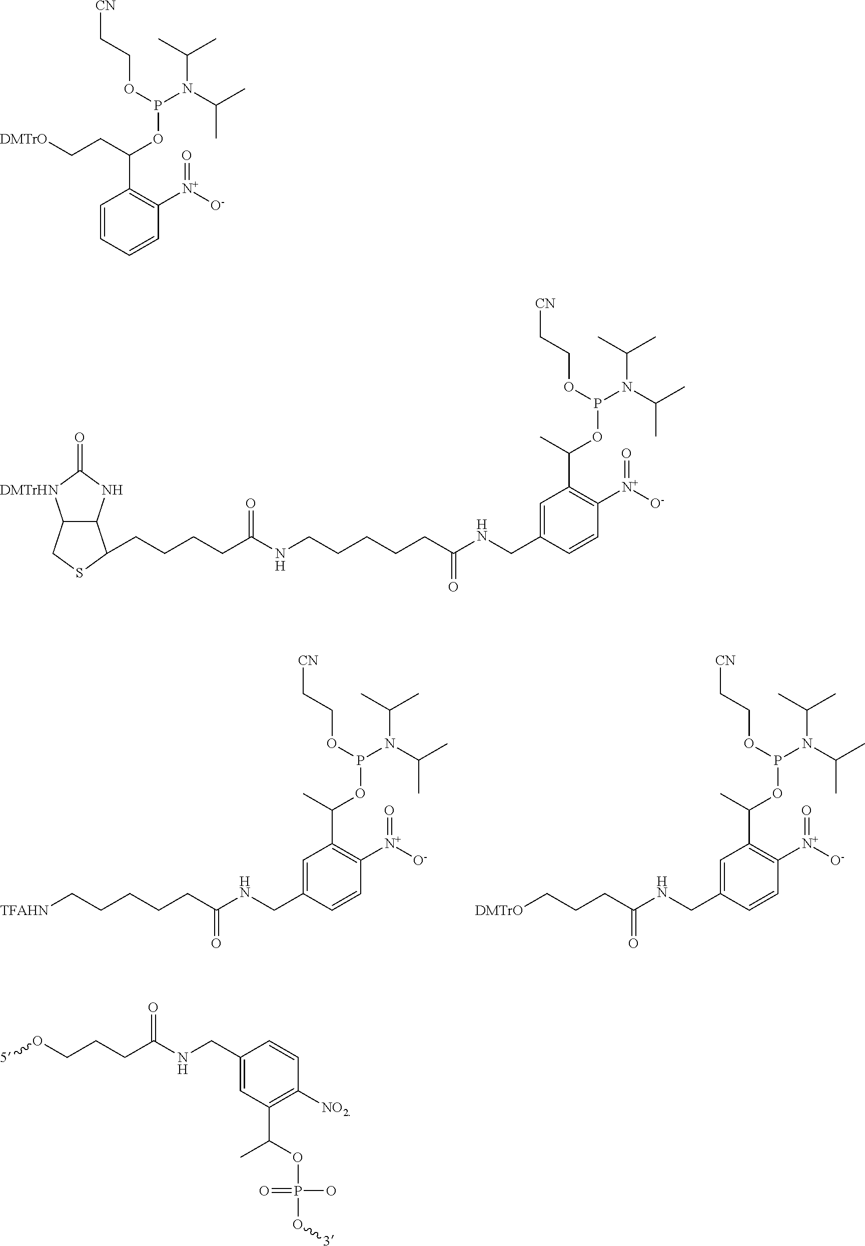

In certain embodiments, when light (e.g., ultraviolet light) is applied, the photocleavable linker may be cleaved, thereby allowing the blocking group to leave, and thus permitting additional nucleic acids to be attached to the DNA tag or other nucleic acid. In some cases, the photocleavable linker may be one which, when reacted by light, causes the exposure of a 5′ phosphate group, which can facilitate the attachment of additional nucleic acids.

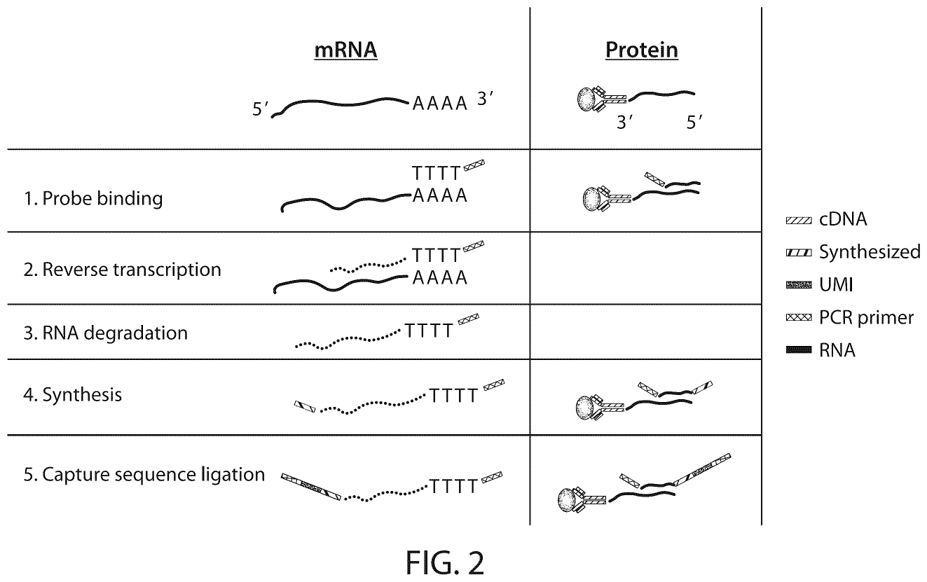

FIG. 2 illustrates one non-limiting example for introducing DNA tags into a sample. In this figure, DNA tags are attached to mRNAs by using the 3′ end poly-A of the mRNAs to attach a poly-T DNA tag onto it. Reverse transcription may then be used to copy the mRNA content onto the DNA tag. A variety of reverse transcriptases are commercially available. The RNA may then be digested, e.g., using RNAse H, leaving the copied DNA tag.

In situ enzymatic DNA synthesis may be performed as follows, in accordance with one embodiment. The DNA synthesis can be performed using a DNA polymerase, for example, TdT. TdT is a naturally occurring polymerase that has the capacity to indiscriminately add single nucleotides to single-stranded DNA. However, the enzyme does not stop at one addition, and can be hard to control the rate of addition under certain conditions. In some cases, a photocleavable linker, such as a Mal-NHS carbonate ester linker can be used to attach a propargylamino-dNTP to the four cysteine residues of the TdT enzyme. When the TdT enzyme adds the nucleotide. the linker keens it in place and physically restricts the addition of more nucleotides. Accordingly, the linker is an example of a blocking group that can be used to prevent the addition of more nucleotides.

In some embodiments, after exposure to the additional nucleic acids, the sample can be washed to remove any free-floating nucleic acids or nucleotides that have not been attached to the sample. For example, in some aspects, after appending, the remaining unbound DNA letters or other nucleic acids may be removed. For example, saline, formamide, or other fluids may be used to remove the remaining unbound DNA letters or other nucleic acids.

Next, if another cycle of DNA letter addition is needed, the DNA letters may be added to the sample and ligation or synthesis may occur in locations defined by light in accordance with certain embodiments, e.g., as discussed herein. The photocleavable linker can be cleaved using light, e.g., UV light, to allow for additional nucleic acids to be attached, e.g., as described above. This can then be used to deterministically add nucleotides or other nucleic acids to the DNA tag or other nucleic acids within the sample. As noted, this may be repeated in certain embodiments any suitable number of times, e.g., depending on the number of DNA letters or other nucleic acids that may be used to form barcodes.

As another example, ligation-based DNA barcoding can be used in various embodiments. For example, ligation of photocleavable oligonucleotides can be used to introduce spatial barcodes. In this case, some or all of the DNA letters or other nucleic acids in a barcode may comprise a sequence of nucleotides, which may be linked to a spacer sequence, e.g., by a photocleavable linker.

Such methods of barcode creation can be understood from a recursive algorithm in some cases. A non-limiting example is depicted in FIG. 5. This example starts from at least DNA letters, Bᵢ and Bⱼ, already ligated onto the sample, with the DNA letter Bⱼ having a photocleavable spacer. In the spatial locations where the next DNA letter would be added, light (e.g. UV light) is applied to cause cleavage of the spacer sequence and expose a 5′ phosphate group. This would make the oligonucleotides at those locations ready for ligation. In other locations that were not photocleaved, there would be no 5′ phosphate group at the 5′ end of the oligonucleotides and those oligonucleotides would not participate in ligation. Accordingly, by controlling where light is applied, different nucleic acids can be added to different portions of a sample.

In one set of embodiments, the attachment of nucleic acids may be facilitated through the use of splints. The splint may have, for example, a first portion and a second portion, wherein the first portion binds to at least a portion of a first nucleic acid and the second portion sequence binds to at least a portion of a second nucleic acid. For example, in some embodiments, a mixture of splints containing some or all possible combinations of complements to the previous DNA letters or other nucleic acids (Bᵢ and Bⱼ in this example) can then be applied, and an overhang corresponding to the complement of the next letter (e.g., Bₖ in this example). A fraction of the splints that match the right letter complements may be annealed onto the previous letters. The next DNA letter linked to a spacer sequence with a photocleavable linker can be ligated on using, for example, T4 DNA ligase, or other suitable ligases. In some embodiments, the 3′ ends of the splint oligonucleotide may be blocked with a blocking group (for example, by a C3 spacer), e.g., to prevent it from ligating onto the oligonucleotides. The splints can be detached, for example, using formamide or other suitable techniques, and the splints can be washed away (e.g., using saline, etc. as described herein), to make the system ready for the next round of nucleic acid (or “bit”) addition. This process can be repeated in certain cases to continue to add DNA letters or other nucleic acids, e.g., to form a barcode.

Another aspect is generally directed to spatial light addressing. In some embodiments, one or more locations can be defined within a sample as locations where light, e.g., beams of light, are directed. In some cases, specific locations can be defined as pixels or voxels, e.g., where two, three, four, or more beams of light are directed. The pixels or voxels may, in some embodiments, have the same or different shapes and/or sizes, and may be defined in two or three dimensions within the sample.

In some cases, the pixels (if present) may have an average area of less than 1 mm², less than 0.5 mm², less than 0.3 mm², less than 0.1 mm², less than 0.05 mm², less than 0.03 mm², less than 0.01 mm², less than 0.005 mm², less than 0.003 mm², less than 0.001 mm², less than 500 micrometers², less than 300 micrometers², less than 100 micrometers², less than 50 micrometers², less than 30 micrometers², less than 10 micrometers², less than 5 micrometers², less than 3 micrometers², less than 1 micrometers², etc.

In addition, in some cases, the voxels (if present) may have an average volume of less than 1 mm³, less than 0.5 mm³, less than 0.3 mm³, less than 0.1 mm³, less than 0.05 mm³, less than 0.03 mm³, less than 0.01 mm³, less than 0.005 mm³, less than 0.003 mm³, less than 0.001 mm³, less than 0.0005 mm³, less than 0.0003 mm³, less than 0.0001 mm³, less than 0.00005 mm³, less than 0.00003 mm³, less than 0.00001 mm³, less than 5000 micrometers³, less than 3000 micrometers³, less than 1000 micrometers³, less than 500 micrometers³, less than 300 micrometers³, less than 100 micrometers³, less than 50 micrometers³, less than 30 micrometers³, less than 10 micrometers³, less than 5 micrometers³, less than 3 micrometers³, less than 1 micrometers³, etc.

In some embodiments, the pixels or voxels may be defined by beams of light that are applied substantially orthogonally to each other, e.g., forming a square or rectangular grid of pixels or voxels (in 2 or 3 dimensions, respectively). However, in other embodiments, the beams of light may be applied at angles that are not substantially orthogonal to each other. In some cases, the beams of light may be applied to form pixels or voxels that are rhombuses or other nonrectangular parallelograms (in 2 or 3 dimensions, respectively). In certain cases, some or all the beams of light may be applied from one side of a sample, and applied at different angles.

The light map can be applied in parallel across pixels or voxels, in certain embodiments. For example, in a round of synthesis, locations that have the same DNA letter may be simultaneously addressed, e.g., to parallelize the synthesis step. A variety of methods can be used to control the application of light to a sample, and this can be done in 2 or even 3 dimensions in some embodiments.

In one non-limiting example of a 2-dimensional approach, a spatial light modulator, such as a DMD projector, can be used to create a grid of pixels. In some cases, the pixels can have a dimension less than 100 micrometers, less than 75 micrometers, less than 50 micrometers, less than 40 micrometers, less than 30 micrometers, less than 25 micrometers, less than 20 micrometers, less than 15 micrometers, less than 10 micrometers, less than 8 micrometers, less than 6 micrometers, less than 5 micrometers, less than 4 micrometers, less than 3 micrometers, less than 2 micrometers, less than 1 micrometer, less than 0.5 micrometer, less than 0.2 micrometer or other suitable dimensions. To parallelize addressing the pixels, the pixels may be activated for all of the locations or regions that correspond to the same DNA letter. The photocleavable linker can be cleaved using light, such as UV light, which is achievable using commercial LED or laser options, or other suitable light sources. Examples of spatial light modulators (SLMs) that may be used include, but are not limited to, liquid crystal on silicon (LCOS) chips, digital micromirror devices (DMDs), acousto-optic deflectors (AOD), etc.

In addition, a variety of methods can be used to produce 3D pixels or “voxels,” e.g., such that the locations where light is applied is defined in 3-dimensional space. The light may be applied from one location or more than one location. In some cases, light may be applied to a sample from a location “above” the sample, e.g., applied at different angles such that each location is defined by the intersection of 3 different beams of light, thereby identifying a unique location within the sample.

For example, the light may be applied form a single side of the sample. As is shown in FIG. 11, for example, one way to achieve this is to have sheets of light perpendicular to y, z+x, or z-x axes. It should be understood that the various beams of light need not intersect a sample at substantially orthogonal directions (although they can be), e.g., as is shown in FIGS. 11A-11C.

Such a configuration can be used with a large sample and thickness. This allows thicker samples to be studied in certain embodiments, e.g., samples such as tissues that are 1 mm thick and several mm in length and width. For instance, the sample may have a thickness of at least 0.01 mm, at least 0.05 mm at least 0.1 mm, at least 0.3 mm, at least 0.5 mm, at least 1 mm, a least 1.3 mm, at least 1.5 mm, at least 2 mm, at least 2.5 mm, at least 3 mm, at least 4 mm, at least 5 mm, etc. This may include samples such as small organs and organisms. For example, a mouse embryo after organogenesis is 5 mm×5 mm×2 mm in size, or a melanoma model in zebrafish is a −2 mm in each dimension.

Yet another aspect is generally directed to various techniques for error detection and/or error correction, e.g., for spatial barcoding, barcode generation, sequencing, or the like. Although not necessarily required, in certain embodiments, e.g., with relatively large samples, numbers of barcodes, etc., error correction can be important to reduce errors in accordance with certain embodiments. In some aspects, the DNA letters and/or the barcodes may be used to define an error-detecting and/or an error-correcting code, for example, to reduce or prevent misidentification or errors of the nucleic acids. Such error-detecting and/or the error-correction code may take a variety of forms.

In one set of embodiments, repeat codes may be used, e.g., where a DNA letter or other nucleic acid sequence is repeated 2, 3, 4, 5, or more times. Repeat codes may allow for error detection for any number of repeats and for error correction for 3 or more repeats. As an illustrative non-limiting example, if a barcode contains two different DNA letters from a set of 4 DNA letters (A, B, C, and D), a 2-repeat code would have the following possibilities: AABB, AACC, AADD, BBAA, BBCC, BBDD, CCAA, CCBB, CCDD, DDAA, DDBB, DDCC. For an independent one-nucleotide error (e.g., AABB become ACBB), the error can be determined by detecting a break in the repeat.

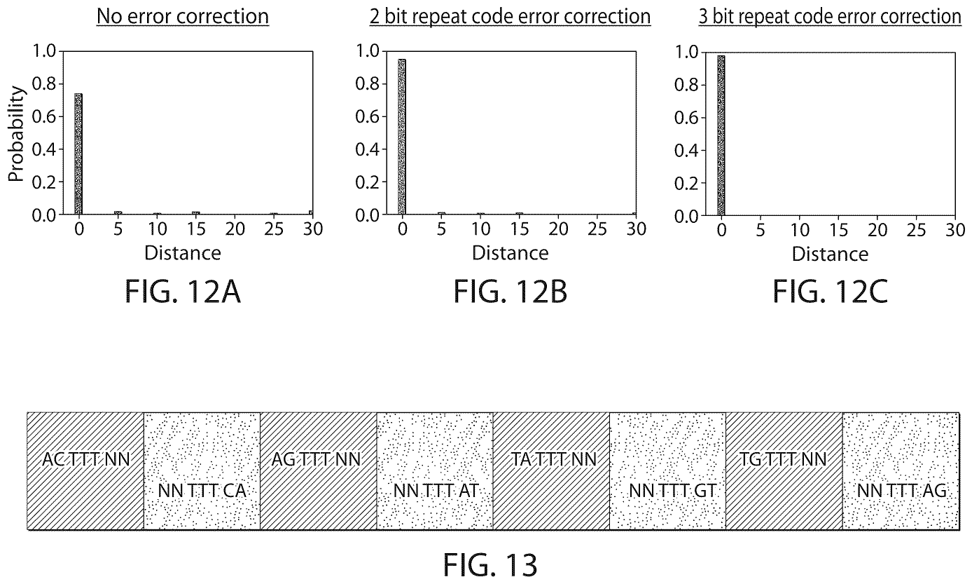

However, in this particular example, this could not be corrected since it is ambiguous whether the correct sequence was AABB or CCBB if the sequence was ACBB. In a 3-repeat code, a majority can be used to correct for errors. As a non-limiting example, if AACBBB was detected, there would be a break in the AAC repeat, and since two of the three repeats were A, the majority can be used to determine that the letter there should have been A. As a non-limiting example, FIGS. 12A-12C show that for a 10-DNA letter long spatial barcode, the overall fidelity would be about 75% from synthesis errors of 3% per round without error correction. The figure is a Monte-Carlo simulation showing the probability distribution of the distance between the inferred location and the actual location of barcodes. For a 3-time repeat code, this fidelity may be over 99% in some embodiments.

In another set of embodiments, the DNA letters and/or the barcodes may be assigned within a code space such that the assignments are separated by a Hamming distance, which measures the number of incorrect “reads” in a given pattern that cause the codeword or the associated spatial location to be misinterpreted as a different valid codeword or spatial location In certain cases, the Hamming distance may be at least 2, at least 3, at least 4, at least 5, at least 6, or the like. In addition, in one set of embodiments, the assignments may be formed as a Hamming code, for instance, a Hamming(7, 4) code, a Hamming(15, 11) code, a Hamming(31, 26) code, a Hamming(63, 57) code, a Hamming(127, 120) code, etc. In another set of embodiments, the assignments may form a SECDED code, e.g., a SECDED(8,4) code, a SECDED(16,4) code, a SCEDED(16, 11) code, a SCEDED(22, 16) code, a SCEDED(39, 32) code, a SCEDED(72, 64) code, etc. In yet another set of embodiments, the assignments may form an extended binary Golay code, a perfect binary Golay code, or a ternary Golay code. In another set of embodiments, the assignments may represent a subset of the possible values taken from any of the codes described above. The error-correcting code may be a binary error-correcting code, or it may be based on other numbering systems, e.g., ternary or quaternary error-correcting codes (which may be useful if 4 bases are used to define the DNA tags). For instance, in one set of embodiments, more than one type of signaling entity may be used and assigned to different numbers within the error-correcting code.

In addition, some errors can be correlated such as those caused by cross-talk between neighboring spots where beams of light cross. For instance, some errors may be caused by “cross-talk” between neighing spots where beams of light cross, e.g., due to spreading from Gaussian or Rayleigh scattering. However, since the intensity of sites 2 pixels or voxels away may be practically zero, or at least relatively low, in one embodiment, a “mosaic” may be created where first “even” sites are tagged. In this case, there is some cross-talk with the “odd” sites, which may result in unwanted appending or attachment in some embodiments. Next, a universal “stopper” sequence (e.g., such as TTT) can be used, and then the “odd” sites are tagged (whereas “even” sites may then result in unwanted appending or attachment). However, because of the presence of the “stopper” sequences, any sequenced nucleotides before the stopper sequence in the “odd” sites and sequenced nucleotides after the stopper sequence in the “even” sites can be identified and ignored as being due to unwanted appending.

It should be understood that a wide variety of sequences may be used as stopper sequence, e.g., positioned between “even” or “odd” sites. For instance, a stopper sequence may have a length of at least 1, at least 2, at least 3, at least 4, at least 5, at least 6, at least 7, at least 8, at least 9, at least 10, at least 12, at least 15, at least 20, at least 25, at least 30, at least 35, at least 40, or at least 50 nucleotides in length. In addition, the stopper sequence may have a length of no more than 50, no more than 40, no more than 35, no more than 30, no more than 25, no more than 20, no more than 15, no more than 12, no more than 10, no more than 9, no more than 8, no more than 7, no more than 6, no more than 5, no more than 4, no more than 3, or no more than 2 nucleotides. Combinations of any of these are also possible in certain embodiments.

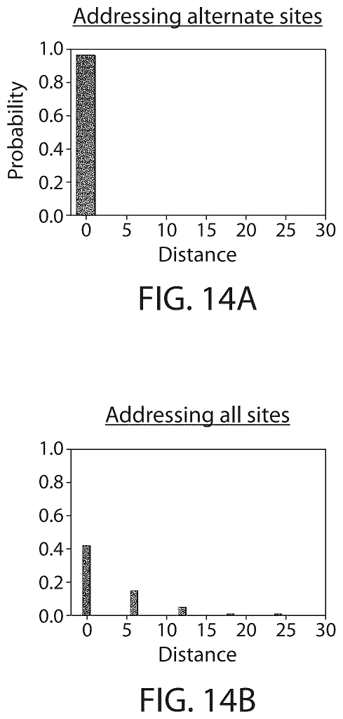

See FIG. 13 for a non-limiting example of this approach. One simulation of such an approach is shown in FIG. 14, as a non-limiting example. In this example, it was found that if every alternate site is addressed, the distance between the inferred position and real position is 0 over 97% of the time. On the other hand, if alternate sites and stopper sequence are not used, the distance is 0 only 42% of the time.

In certain aspects, the DNA tags or other nucleic acids, in some cases barcodes, may be extracted or removed from a sample, and sequenced in certain instances. The DNA, barcodes, or other nucleic acids may be removed from the sample and sequenced using any suitable technique known to those of ordinary skill in the art. Examples of DNA sequencing techniques include, but are not limited to, PCR (polymerase chain reaction), “sequencing by synthesis” techniques (e.g., using DNA synthesis by DNA polymerase to identify the bases present in the complementary DNA molecule), “sequencing by ligation” (e.g., using DNA ligases), “sequencing by hybridization” (e.g., using DNA microarrays), nanopore sequencing techniques, or the like. Optionally, the extracted nucleic acid sequences may be amplified, duplicated, or expanded by PCR, rolling circle replication, or other techniques known to those of ordinary skill in the art. As a non-limiting example, in some cases, a PCR primer may be added or ligated to the barcodes, which subsequently may allow PCR amplification and/or ligation of sequencer-specific adapters for sequencing.

Each of the following is incorporated herein by reference in its entirety: U.S. Provisional Patent Application Ser. No. 63/506,337, filed Jun. 5, 2023, entitled “Chemical Ligation Techniques,” by Zhuang, et al.; U.S. Provisional Patent Application Ser. No. 63/458,610, filed Apr. 11, 2023, entitled “Multiplexed Optical Barcoding for Spatial Omics,” by Zhuang, et al.; and U.S. Provisional Patent Application Ser. No. 63/506,294, filed Jun. 5, 2023, entitled “Multiplexed Optical Barcoding for Spatial Omics,” by Zhuang, et al.

The following examples are intended to illustrate certain embodiments of the present disclosure, but do not exemplify the full scope of the disclosure.

### Example 1

As a non-limiting example, an overhanging sequence that is 5 bases long and with at least 40% GC content has close to 100% ligation efficiency within 10 minutes. The melting temperature of splints that have two letters with 5 bases and 40% GC content is >25° C. and this example illustrates that these can be annealed during ligation and removed with a 30% formamide solution after the ligation is completed. Through this recursive mechanism, barcodes for spatial location multiplexing can be prepared.

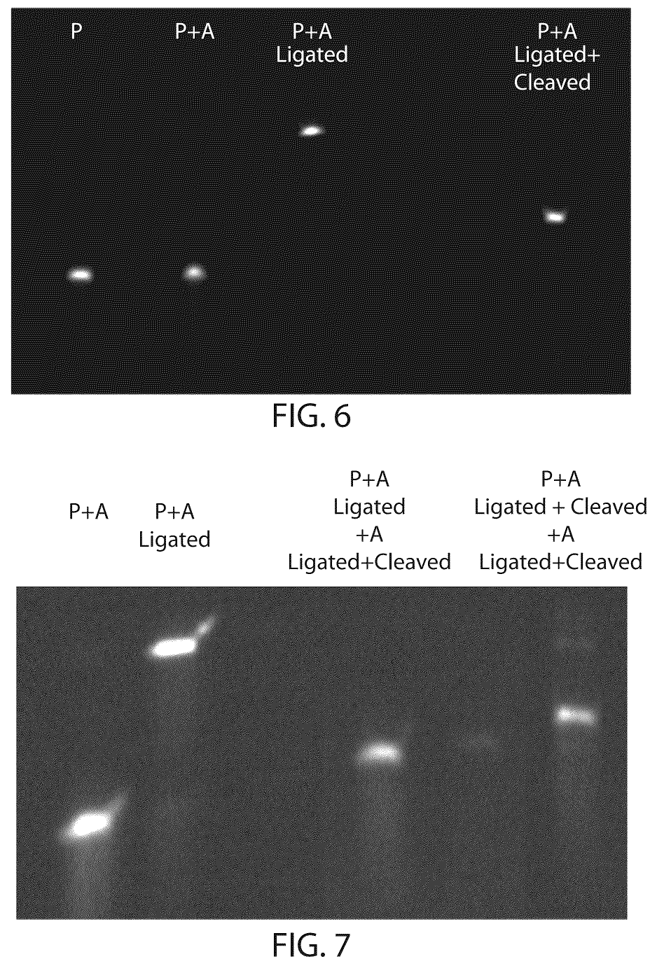

The following example illustrates the approach of ligation and photocleaving in vitro in a test tube. A DNA tag (P=/5Phos/AGAGA TGTGT TGTGT T(10)/36-FAM/(SEQ ID NO: 54)) that has a fluorophore in its 3′ end may be used. A DNA letter with a photocleavable spacer (A=T(25) (SEQ ID NO: 55)/iSpPC/AGAGA) may be ligated using a splint (S=ACACA TCTCT TCTCT (SEQ ID NO: 56)) using Quick Ligation™ Kit (vendor: New England Biosciences) and the products of the reaction run on a 15% TBE (tris borate EDTA) urea denaturing gel. 3 pMol of the DNA tag, 30 pMol of the DNA letter, 30 pMol of the splint and 1 microliter of the Quick Ligase™ Enzyme were used in a 20 microliter reaction for 10 minutes at room temperature. The DNA tag, letter, and splints were ordered from IDT and diluted to 100 micormolar in nuclease free water and stored at −20° C. After 10 minutes, the reaction products were purified in 10 microliters of nuclease free water using Monarch DNA Cleanup kit from NEB before running on the gel. This assay allows for the quantification of the ligation and photocleaving efficiency directly by observing the location of the fluorescent bands on the gel. The results of this assay are shown in FIG. 6: column 1 is the primer, column 2 has the primer, letter, and splint but no ligase enzyme, column 3 is the ligated reaction for 5 minutes, column 4 is the ligated reaction followed by 10 minutes of photocleaving using a 365 nm LED at an intensity of 0.1 W/cm². From the band shifts in the gel, the ligation efficiency was estimated at greater than 95% and the photocleaving efficiency was estimated at greater than 95%.

### Example 2

The following example shows ligation of a DNA letter in a second iteration after photocleaving the previous DNA letter. The same DNA tag and DNA letter as Example 1 was used in this assay. The DNA letter was first ligated using splint S and photocleaved as above. For the next ligation reaction, a longer splint (S2=ACACA ACACA TCTCT TCTCT TCTCT (SEQ ID NO: 57)) was used, which preferentially attaches to the primer over the shorter splint.

The products of the ligation reaction from Example 1 after photocleaving were purified into 3 microliters of nuclease free water using Monarch DNA Cleanup kit from NEB. 3 microliters of 20 micromolar splint (S2) and 3 microliters of 10 uM DNA letter were added along with 1 microliter Quick Ligase™ Enzyme in a 20 microliter reaction for 10 minutes at room temperature. After 10 minutes, the reaction products were purified in 10 microliter nuclease free water using Monarch DNA Cleanup kit from NEB before running on the gel.

FIG. 7 shows the results of this assay wherein column 1 is the unligated primer and letter, column 2 is the ligated primer and letter, column 3 is the second round of ligation without photocleaving the first round, column 4 is the second round of ligation after photocleaving the first round. From the size shifts, it can be estimated that the second ligation reaction also occurred with efficiency of greater than 95%. In addition, it was also shown that there was no further ligation reaction without photocleaving.

### Example 3

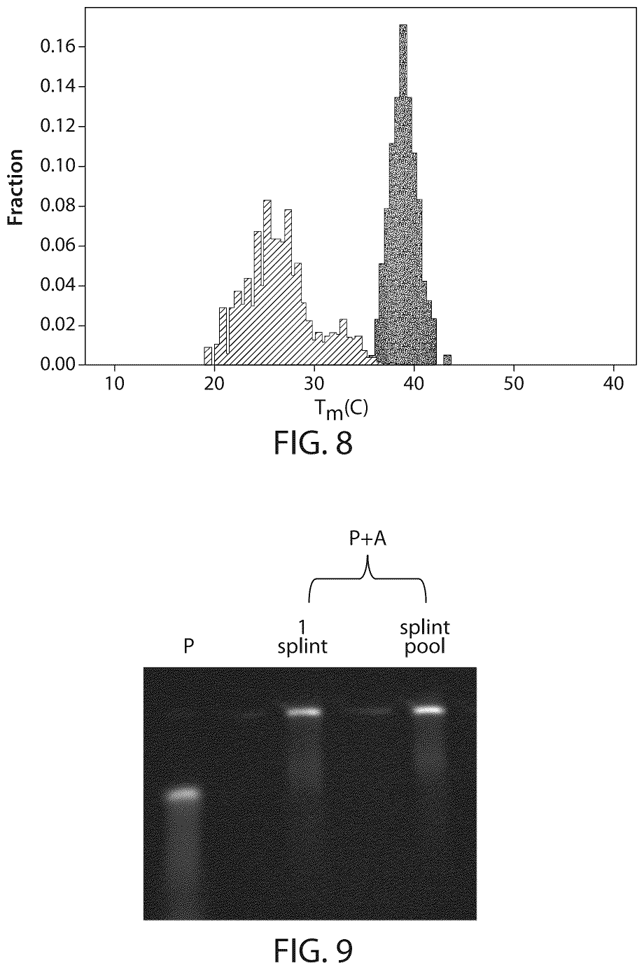

This example illustrates that a pool of splints can be used in order to have spatial multiplexing as discussed in the above examples. The first step in this process was to design a set of letters and splints allowing the addition of the next letter efficiently when a pool of splints is present. Simulations were run over various choices letters containing 5 bases and a 40% GC content such that the melting temperature of a splint with the correct complementary sequence (right histogram in FIG. 8) is distinct from the melting temperature of a splint with one letter mismatch (left histogram).

The results are shown in FIG. 8 where the melting temperature of the wrongly matched splints are centered at room temperature and would constantly detach off the primer until the correct splint, whose melting temperature is much higher than room temperature, anneals on. The ligation assay was repeated as before using DNA tag (P=/5Phos/TGTGT TAGGT ATGGA AGAGA T(10)/36-FAM/(SEQ ID NO: 58)) and DNA letter (A=T(25) (SEQ ID NO: 55)/iSpPC/AGAGA) using either a splint with correct complementarity or a splint with all possible prior letter combinations. The concentration and reaction conditions are the same as Example 1. In the case of the splint pool, 3 microliters of 10 micromolar concentration per splint was used. The reaction products were column purified as before and run on a 15% TBE (tris borate EDTA) urea denaturing gel. The results are shown in FIG. 9 and shows that the ligation efficiency was just as good (greater than 95%) using a splint pool.

### Example 4

The above reactions were tested in-vivo in cultured U2OS cells in this example. The cells were plated onto Ibidi p-Slide VI 0.4 fluidics chamber with ibiTreat coating and cultured overnight. The cells were fixed for 10 minutes with 4% PFA and permeabilized with 0.25% Triton X-100 in PBS for 10 minutes. The sample was then washed with PBS. A DNA Tag (P=/5Phos/TGTGT TAGGT ATGGA AGAGA T(45) (SEQ ID NO: 59) R2, R2 is a PCR primer) is annealed onto the poly-A tail of RNA molecules inside cells using the following buffer: 2 microliters of 25% Triton X-100, 1 microliter of 100 micromolar DNA Tag, 6 microliters of RNAse inhibitor, 20 microliters of 5×RT reaction buffer, and 71 microliters of H₂O. A DNA letter (A1=T(25) (SEQ ID NO: 55)/iSpPC/AGAGA) was ligated using the splint pool from example 3 and photocleaved. 1 microliter of 50 micromolar A1, 1 microliter of 50 micromolar per splint in splint pool, 1 microliter of Quick Ligase™ Enzyme, 22 microliters of nuclease free water in a 50 microliter reaction was used, followed by incubation for 10 minutes at room temperature. The chamber was washed twice with a 0.2×SSC, 50% formamide solution and then with PBS. The sample was photocleaved for 10 minutes using a 365 nm LED at an intensity of 0.1 W/cm². Another DNA tag (A2=T(25) (SEQ ID NO: 55)/iSpPC/ATGGA) was ligated using the corresponding splint pool and photocleaved as in the previous round. Finally a PCR primer (R1) was ligated as in the previous rounds. The oligonucleotides were extracted by degrading the RNA using RNAseH (1.5 microliters RnaseH from NEB in thermopol buffer and 50 microliters reaction volume). The extracted oligonucleotides were PCR amplified and run on Invitrogen™ E-Gel™ EX Agarose Gels, 4% to determine size. Only the extracted oligonucleotides with the PCR primer R1 were amplified.

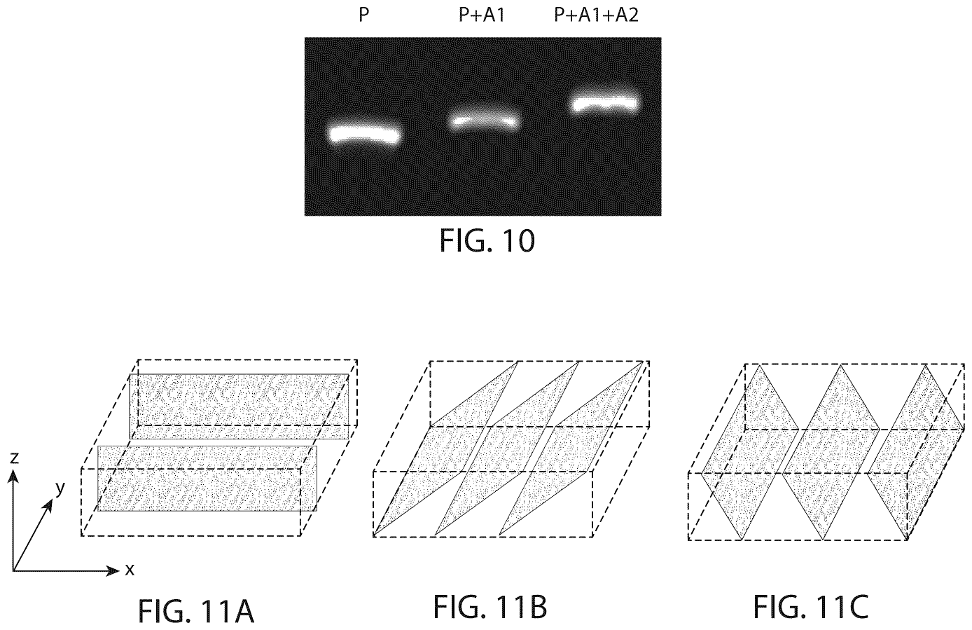

Suppose the efficiency of in-vivo ligation is el and the combined efficiency of photocleaving and washing is ew. In the assay where only A1 was ligated with R1, the intensity ratio of the gel band corresponding to one letter addition A1 compared to no letter addition would be (el*ew)/(1−el). In the assay where A1, A2, and R1 were ligated, the intensity ratio of the gel band corresponding to two letter addition compared to one letter addition would be (el*ew)/(2−el−ew). This allows for the determination of el and ew from FIG. 10 in vivo in cultured cells to be greater than 98% each.

### Example 5

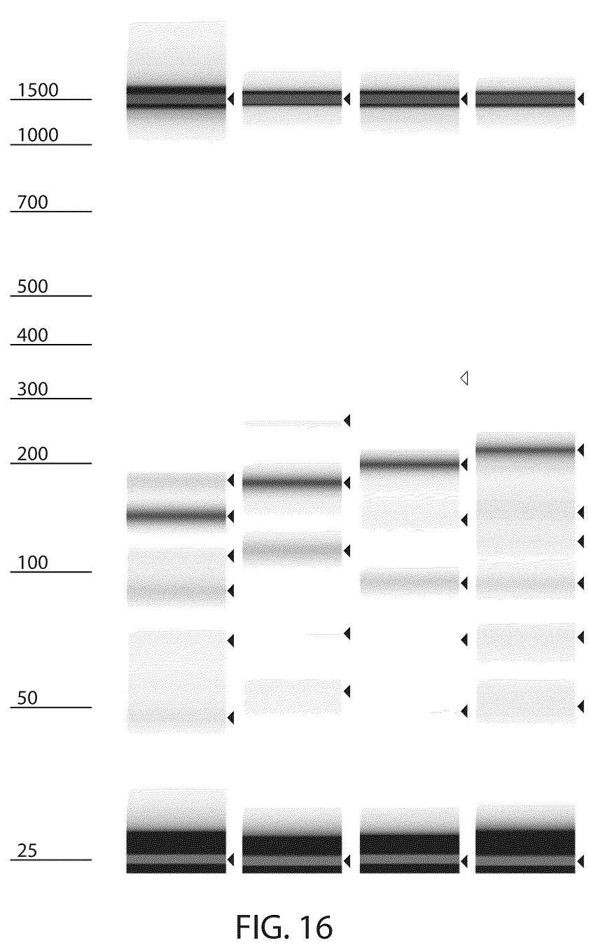

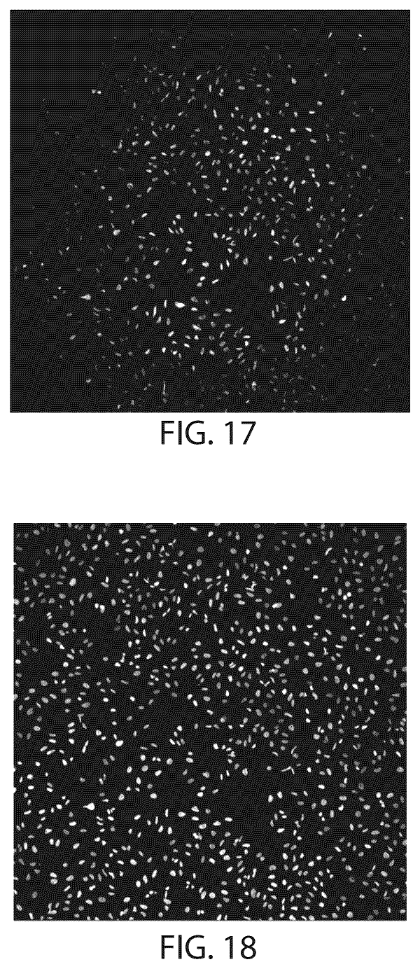

This example demonstrates >95% efficiency per photosensitive ligation over 4 ligations in cultured U2OS cells. The cells were plated in a Ibidi p-Slide VI 0.4 coated with poly-d-lysine and grown overnight. The cells were fixed for 10 minutes with 4% PFA and washed with 1×PBS. A DNA tag (/5Phos/AGAGA ATGGA TAGGT TGTGT AATCAGCCATACCACATTTG TT+TT+TT+TT+TT+TT+TT+TT+TT+TT+TT+TTT (SEQ ID NO: 60)) was hybridized onto the poly-A tail of RNA molecules inside the cells/tissue at 1 μM concentration in 2×SSC at 37 C overnight. The samples were washed two times with 30% formamide in 2×SSC at 47 C for 30 mins to remove any unspecifically bound DNA tag. 4 different DNA letters were used (L1=T(25) (SEQ ID NO: 55)/iSpPC/AGAGA ATGGA TAGGT TGTGT (SEQ ID NO: 61), L2=T(25) (SEQ ID NO: 55)/iSpPC/ATGGA AGAGA TGTGT TAGGT (SEQ ID NO: 62), L3=T(25) (SEQ ID NO: 55)/iSpPC/TAGGT TGTGT AGAGA ATGGA (SEQ ID NO: 63), L4=T(25)(SEQ ID NO: 55)/iSpPC/TGTGT TAGGT ATGGA AGAGA (SEQ ID NO: 64)). Letter L2 is first ligated onto the DNA tag using a splint (S2=TCCAT TCTCT ACCTA (SEQ ID NO: 21)) in the following buffer: 50 μl 2× ligation buffer, 42 μl H2O, 2 μl 40 μM letter, 2 μl 40 μM splint, 2 μl 25% TX-100, 2 μl Quick Ligase, where the 2× ligation buffer was made fresh with 132 mM Tris, 20 mM MgCl2, 2 mM ATP, 2 mM DTT, 15% PEG 8000 adjusted to pH 7.6 with 1M HCl. The ligation reaction was carried out at room temperature for 1 hour. The sample was photocleaved for 1 minute using a 365 nm LED at 0.1 W/cm2. The sample was subsequently ligated with L1 using a splint (S1=TCTCT TCCAT ACACA (SEQ ID NO: 29)). The sample was photocleaved again and ligated with L3 using a splint (S3=TCCAT TCTCT TCCAT (SEQ ID NO: 65)). The sample was photocleaved again and ligated with L4 using a splint (S4=ACACA ACCTA TCTCT (SEQ ID NO: 12)). The samples were then heated to 93 C for 3 minutes in water and the water containing the DNA tags was aspirated out. The solution was annealed with the sequence complementary to AATCAGCCATACCACATTTG (SEQ ID NO: 66) in the DNA tag and run on the HS DNA 1000 tape on an Agilent 4200 Tapesatation. The size observed on the tapestation is reflective of the DNA letters being ligated onto the tags and the efficiency of the ligations and photocleaving can be estimated from the relative height of the correct peak to all the other peaks. FIG. 16 shows the tapestation results for 1, 2, 3, and 4 ligations, with >95% efficiency per ligation.

### Example 6

This example demonstrates spatial selectivity of photocleaving using a Digital Mirror Device (DMD), wherein a fluorescent signal was used that only appears in the region that was photocleaved. Cultured cells were used, prepared as in the previous example, using the same DNA tag and ligated first with L2. Polygon1000-G from Mightex was used in a Olympus IX71 microscope body with a Nikon 10× plan apo λ objective and a region of 1 mm×0.6 mm was illuminated at 365 nm LED at 0.1 W/cm2 for 1 minute. A letter (L1-sty=GAT CCG ATT GGA ACC GTC CC (SEQ ID NO: 67)/iSpPC/AGAGA ATGGA TAGGT TGTGT (SEQ ID NO: 61)) was then ligated and the presence of L1-sty was detected using a fluorescently tagged conjugate of the sequence GAT CCG ATT GGA ACC GTC CC (SEQ ID NO: 67) in L1-sty. FIG. 17 shows the fluorescent signal in the photocleaved region while FIG. 18 is a DAPI signal showing the presence of cells everywhere.

### Example 7

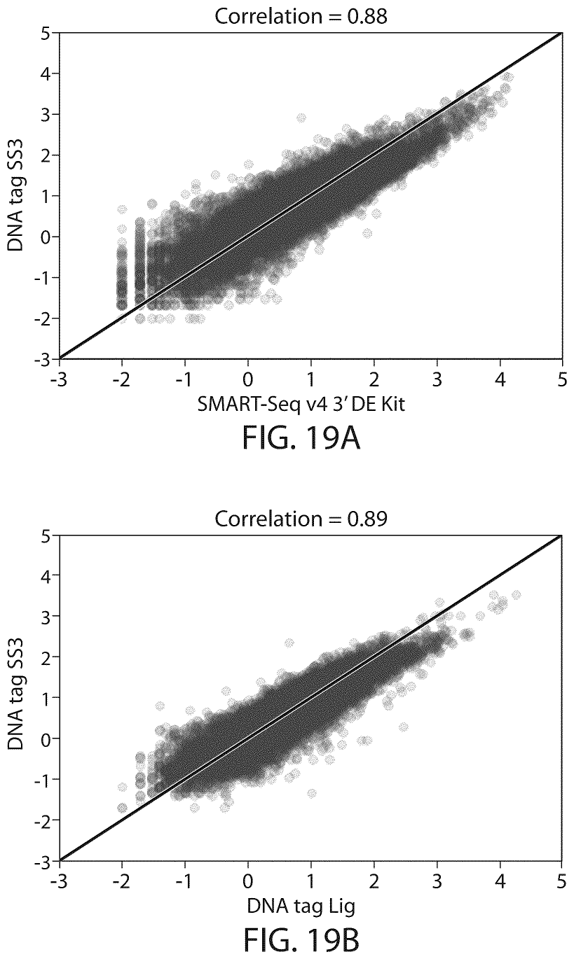

This example demonstrates that the mRNA content of tissue samples can be reliably captured using in-situ Reverse Transcription (RT), including with the ligation chemistry disclosed herein. To demonstrate this, Mouse brain cortex tissue was used to prepare three samples. In one of the sections, RNA was extracted and a sequencing library was prepared using SMART-Seq v4 3′ DE Kit. In another section, it was hybridized with DNA tag (SS3=ACGAGCATCAGCAGCATACGA NNNNNNNNNNNNNNNNNNNN TTTTTTTTTTTTTTTTTTTTTTTTTTTTTT VN (SEQ ID NO: 68)) and in another section it was hybridized with DNA tag (Lig=/5Phos/AGAGA ATGGA TAGGT TGTGT NNNNNNNNNNNNNNNNNNNN TTTTTTTTTTTTTTTTTTTTTTTTTTTTTTVN (SEQ ID NO: 69)). After overnight hybridization, the samples were washed twice with 2×SSC and briefly with water and put into RT buffer (7.5 μl 25 mM dNTP, 25 μl 5×RT buffer, 1.5 μl Rnaseln, 1.5 μl Rnase inhibitor 40 U/μl, 2.5 μl 100 μM TSO, 5 μl Maxima H minus 200 U/μl, and 57 μl H2O, where the TSO was /5Biosg/AAGCAGTGGTATCAACGCAGAGTACATrGrG+G (SEQ ID NO: 70)). The samples were incubated at 37 C overnight. The following day, the samples were washed twice with 2×SSC and twice with 1×PBS. For the sample with DNA tag Lig, the sequence ACGAGCATCAGCAGCATACGA (SEQ ID NO: 73) was ligated. cDNA from both samples was extracted in 1×Seqamp CB buffer with 1.5 ul of Rnase H in 100 ul total volume at 37 C for 1 hour. The extracted samples were PCR for 8 cycles and then tagmented using the Illumine Nextera XT kit and 5′ enriched using the PCR primers GTCTCGTGGGCTCGGAGATGTGTATAAGAGACAG (SEQ ID NO: 71) and TCGTCGGCAGCGTCAGATGTGTATAAGAGACAG ACGAGCATCAGCAGCATACGA (SEQ ID NO: 72). The samples were indexed and then sequenced at Novogene with 20 million reads each. Results are shown in FIGS. 19A-19B.

### Example 8

This example demonstrates the spatial selectivity of barcoding and validating it through sequencing. Two different species of cells, U2OS and MEF were plated next to each other in a Ibidi Culture-Insert 4 Well in μ-Dish coated with Poly-d-lysine. The cells were fixed and hybridized with primer/5Phos/AGAGA ATGGA TAGGT TGTGT NNNNNNNNNNNNNNNNNNNN TTTTTTTTTTTTTTTTTTTTTTTTTTTTTTVN (SEQ ID NO: 69) as described in Example 7. The sample was also reverse transcribed as above.

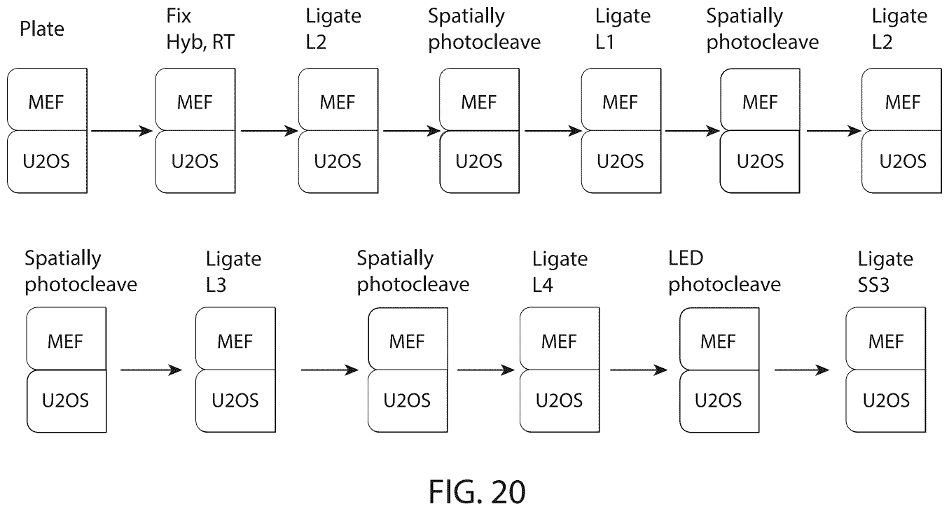

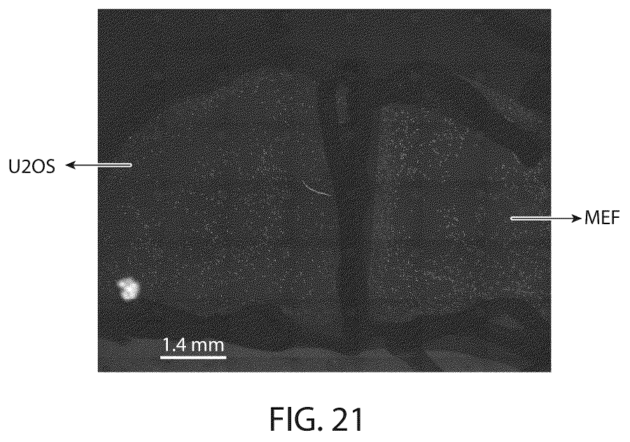

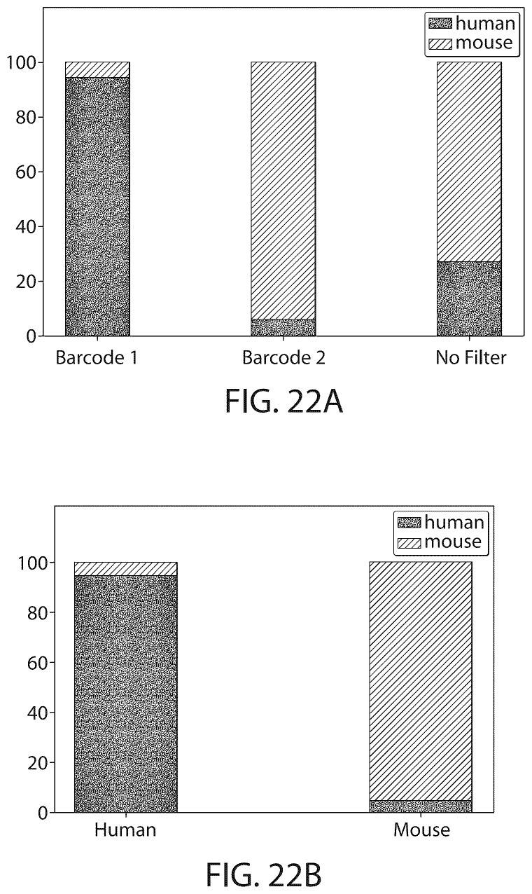

After RT, in this case, the sample was washed twice in 30% formamide 2×SSC at 47 C for 30 mins. After the washes, the divider silicone chamber was removed and the ligation order in FIG. 20 was followed. SS3 was the PCR primer ACGAGCATCAGCAGCATACGA (SEQ ID NO: 73) as in Example 7 and L1, L2, L3, L4 are the same letters as in Example 5. Each sample had two letters ligated in order to build in error-correction and improve the position identification efficiency. FIG. 21 shows a DAPI image of the cells. After the ligations, the cDNA with the DNA tag and barcode was extracted in water at 93 C for 3 minutes. The extracted cDNA was PCRd and followedthe library preparation protocol described in Example 7. The sample was sequenced in Illumina Miseq with 2 million reads. The sequences were split into barcodes containing L1L3 (barcode 1) and L2L4 (barcode 2) and mapped onto the mouse and human genomes. The relative fraction of reads mapping to human and mouse genes are shown in FIG. 22A, showing that ~95% are attributed to the intended species. FIG. 22B shows the sequencing data when the divider was never removed and hence the species were never mixed. Even in this ideal case, there were ~5% genes attributed to the other species.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
applying a first beam of light to a first location in a sample to attach a first nucleic acid to the sample within the first location; and
applying a second beam of light to a second location in the sample to attach a second nucleic acid to the sample within the second location,
wherein the first location and the second location overlap at at least one location within the sample.

2. The method of claim 1, comprising applying a first beam of light to a first set of locations in a sample to attach a first nucleic acid to the sample within the first set of locations.

3. The method of claim 2, comprising applying a second beam of light to a second set of locations in the sample to attach a second nucleic acid to the sample within the second set of locations, wherein the first set of locations and the second set of locations overlap at at least one location within the sample.

4. The method of any one of claims 1-3, comprising attaching the first nucleic acid to a nucleic acid attached to the sample.

5. The method of claim 4, wherein the nucleic acid attached to the sample is covalently attached to the sample.

6. The method of any one of claims 4 or 5, wherein the nucleic acid attached to the sample is non-covalently attached to the sample.

7. The method of any one of claims 4-6, wherein the nucleic acid attached to the sample is attached to RNA.

8. The method of claim 7, further comprising reverse transcribing the RNA.

9. The method of any one of claims 4-8, wherein the nucleic acid attached is linked to an antibody.

10. The method of claim 9, wherein the antibody is configured to recognize a protein.

11. The method of any one of claims 4-10, wherein the nucleic acid attached to the sample is attached to DNA.

12. The method of any one of claims 4-11, wherein the nucleic acid comprises a sequence complementary to an endogenous nucleic acid.

13. The method of any one of claims 4-12, wherein the nucleic acid comprises a plurality of sequences complementary to a plurality of endogenous nucleic acids.

14. The method of any one of claims 4-13, wherein the nucleic acid attached to the sample further comprises a blocking group.

15. The method of claim 14, wherein applying the first beam of light to the sample causes removal of the blocking group from the nucleic acid attached to the sample.

16. The method of any one of claims 1-15, comprising attaching the second nucleic acid to the first nucleic acid.

17. The method of claim 16, further comprising exposing the sample to a DNA ligase able to attach the second nucleic to the first nucleic acid.

18. The method of any one of claims 1-17, further comprising exposing the sample to splint DNA able to bind the first nucleic acid and the second nucleic acid.

19. The method of any one of claims 1-18, further comprising exposing the sample to plurality of splint DNA able to bind the first nucleic acid and the second nucleic acid.

20. The method of any one of claims 1-19, further comprising extracting nucleic acids from the sample.

21. The method of claim 20, comprising sequencing nucleic acids extracted from the sample.

22. The method of any one of claims 1-21, further comprising determining a spatial position of the first nucleic acid and the second nucleic acid.

23. The method of claim 22, further comprising determining a molecular identity of a target within the sample based on the first nucleic acid and the second nucleic acid.

24. The method of any one of claims 22 or 23, comprising determining the spatial position of the first nucleic acid and the second nucleic acid by determining sequences of the first nucleic acid and the second nucleic acid.

25. The method of any one of claims 22-24, wherein the target is an RNA in the sample.

26. The method of any one of claims 22-24, wherein the target is a transcriptome of the sample.

27. The method of any one of claims 22-24, wherein the target is a protein in the sample.

28. The method of any one of claims 22-24, wherein the target is a DNA in the sample.

29. The method of any one of claims 22-28, wherein the spatial positions are encoded by the first nucleic acid and the second nucleic acid.

30. The method of claim 29, wherein the first nucleic acid and the second nucleic acid encode two-dimensional spatial positions.

31. The method of claim 29, wherein the first nucleic acid and the second nucleic acid encode three-dimensional spatial positions.

32. The method of any one of claims 4-31, further comprising determining a spatial position of the nucleic acid attached to the sample.

33. The method of any one of claims 4-32, further comprising determining a molecular identity of a target within the sample based on the sequence of the nucleic acid attached to the sample.

34. The method of any one of claims 32 or 33, comprising determining the spatial position of the nucleic acid attached to the sample by determining sequences of the first nucleic acid and the second nucleic acid.

35. The method of any one of claims 32-34, further comprising determining the molecular identity and the spatial location of the target within the sample based on the sequence of the nucleic acid attached to the sample, the sequence of the first nucleic acid, and the sequence of the second nucleic acid.

36. The method of any one of claims 1-35, wherein the first nucleic acid consists of 1 nucleotide.

37. The method of any one of claims 1-36, wherein the first nucleic acid comprises a plurality of nucleotides.

38. The method of any one of claims 1-37, wherein the second nucleic acid consists of 1 nucleotide.

39. The method of any one of claims 1-38, wherein the first second acid comprises a plurality of nucleotides.

40. The method of any one of claims 1-39, wherein the first nucleic acid comprises a first blocking group.

41. The method of any one of claims 1-40, wherein the first nucleic acid comprises a second blocking group.

42. The method of any one of claims 1-41, wherein the sample comprises cells.

43. The method of any one of claims 1-42, wherein the sample is a single cell.

44. The method of any one of claims 1-41, wherein the sample comprises tissue.

45. A method, comprising:
applying a first beam of light to a first location in a sample to attach a first nucleic acid to the sample within the first location; and
applying a second beam of light to a second location in the sample to attach a second nucleic acid to the sample within the second location such that at at least one spatial position within the sample, the second nucleic acid is attached to the first nucleic acid.

46. The method of claim 45, further comprising applying a third beam of light to a third location of the sample to attach a third nucleic acid to the sample within the third location such that at at least one spatial position within the sample, the third nucleic acid is attached to the second nucleic acid attached to the first nucleic acid.

47. A method, comprising:
applying beams of light to a sample to attach nucleic acids to the sample, each beam of light applied to attach a nucleic acid to attached nucleic acids in the sample to form barcodes comprising a plurality of the nucleic acids.

48. The method of claim 47, wherein the barcodes encode spatial positions where the nucleic acids are attached to the sample.

49. The method of any one of claims 47 or 48, wherein at least some of the barcodes within the sample comprise an error-detection code.

50. The method of any one of claims 47-49, wherein at least some of the barcodes within the sample comprise an error-correction code.

51. A method, comprising:
providing a sample comprising nucleic acids comprising a first sequence, a photocleavable linker, and a spacer sequence;
applying light to a location in the sample to cleave the photocleavable linker and remove the spacer sequence from the nucleic acids in the location; and
attaching a second sequence to the nucleic acid using a DNA ligase, wherein the spacer sequence, when present, inhibits the DNA-ligase from attaching nucleic acids.

52. A method, comprising:
applying beams of light to a sample to attach nucleic acids to different spatial positions within a sample, wherein the nucleic acids at different spatial positions within the sample comprise distinguishable sequences.

53. A method, comprising:
applying beams of light to a sample to attach nucleic acids to different spatial positions within a sample, wherein the nucleic acids encode their spatial positions within the sample.

54. A composition, comprising:
a plurality of nucleic acids, encoding information about their spatial positions within a sample.

55. The composition of claim 54, wherein the plurality of nucleic acids further encodes identities of molecules in the sample.

56. The composition of claim 55, wherein the molecules comprise RNA.

57. The composition of claim 56, wherein the RNA is mRNA.

58. The composition of any one of claims 55-57, wherein the molecules comprise proteins.

59. The composition of any one of claims 55-58, wherein the molecules comprise DNA.

60. A method, comprising:
sequencing nucleic acids taken from a sample; and
determining spatial positions of the nucleic acids within the sample based on spatial information encoded therein.

61. The method of claim 60, further determining identities of molecules in the sample.

62. The composition of claim 61, wherein the molecules comprise RNA.

63. The composition of claim 62, wherein the RNA is mRNA.

64. The composition of any one of claims 61-63, wherein the molecules comprise proteins.

65. The composition of any one of claims 61-64, wherein the molecules comprise DNA.

66. A method, comprising:
sequencing nucleic acids taken from a sample; and
constructing an image based on spatial information encoded within the nucleic acids.

67. A method, comprising:
defining spatial positions within a sample encoded by nucleic acids; and
forming barcodes at the spatial positions within the sample that encodes the spatial positions.

68. A method, comprising:
defining a plurality of spatial positions in a sample as intersections of beams of light applied from at least 2 different angles;
defining distinguishable nucleic acid sequences identifying at least some of the spatial positions; and
forming nucleic acids within at least some of the spatial positions that correspond to the defined nucleic acid sequences that identifies the respective spatial positions.

69. The method of claim 68, wherein the spatial positions are pixels defined as the intersection of two beams of light.

70. The method of claim 68, wherein the spatial positions are voxels defined as the intersection of three beams of light.

71. A composition, comprising:
a first nucleic acid attached to a sample in a first location; and
a second nucleic acid attached to the sample in a second location neighboring the first location,
wherein the first nucleic acid and the second nucleic acid each comprise a stopper sequence between encoding sequences.

72. A method, comprising:
sequencing nucleic acids encoding information about their spatial positions in a sample, wherein the information comprises encoding sequences interspersed with stopper sequences; and
rejecting encoding sequences based on their positions relative to the stopper sequences.
